(19) United States
(12) Patent Application Publication
(10) Pub. No.: US 2023/0263915 A1
(43) Pub. Date: Aug. 24, 2023

(54) RADIOLABELED COMPOUNDS

(71) Applicant: King's College London, London (GB)

(72) Inventors: Ran Yan, London (GB); Filipa Mota, Baltimore, MD (US)

(21) Appl. No.: 18/007,002

(22) PCT Filed: Jul. 28, 2021
(86) PCT No.: PCT/EP2021/071194

§ 371 Date: Jan. 26, 2023

(30) Foreign Application Priority Data
Jul. 29, 2020 (GB) 2011787.5

(51) Int. Cl. A61K 51/04; C07D 277/66

(52) U.S. Cl. CPC A61K 51/0453; C07D 277/66; C07B 2200/05

(57) ABSTRACT

This invention relates to radiolabelled compounds as described herein, precursor compounds and reference compounds, as well as pharmaceutical compositions comprising the radiolabelled compounds, which are for use in a diagnostic method practised on the human or animal body using positron emission tomography (PET).

### TECHNICAL FIELD

This invention relates to radiolabelled compounds, precursor compounds and reference compounds, as well as pharmaceutical compositions comprising the radiolabelled compounds. Aspects of the inventions also relate to the radiolabelled compounds for use in a diagnostic method practised on the human or animal body using positron emission tomography (PET). Further aspects of the invention relate to processes for radiolabelling a precursor compound to form the radiolabelled compound, and processes for making a precursor compound or a reference compound.

### BACKGROUND

Reactive oxygen species (ROS) are generated as by-products of the electron transport chain in living cells. At low levels, and in the presence of endogenous antioxidants, ROS play integral roles in the regulation of cell growth, neurotransmission, and immune responses. However, in elevated levels they lead to the oxidation of DNA, proteins, and lipids, and underlie the pathogenesis of many cardiovascular and neurodegenerative diseases, as well as cancers and inflammatory conditions.

In the cardiovascular system, elevated ROS are responsible for tissue injury during ischemia/reperfusion, and have been linked to the progression from cardiac hypertrophy to heart failure, the evolution of atherosclerotic plaques, and the cardiac and microvascular dysfunction associated with diabetes. ROS production has also been linked to the cardiotoxicity of cancer chemotherapeutic agents, which severely limits their dosimetry and effectiveness. Most notably, the cardiac toxicity induced by Doxorubicin, a widely used cancer chemotherapy agent, has been linked to ROS generation.

A clinically translatable means of noninvasively identifying and quantifying elevated ROS production in vivo would be highly desirable for both diagnostic and prognostic purposes, as well as in the development and evaluation of emerging targeted antioxidant therapies.

Positron emission tomography (PET) is a non-invasive nuclear imaging technique that uses radiolabelled molecules to either detect the expression of a target or monitor a metabolic process in vivo. Some radiolabelled small molecules have been reported to indirectly report on oxidative stress levels, and hydroethydium-based radiotracers have been tested in vivo in rodent models of cardiotoxicity and inflammation. Nonetheless, there are currently no radiotracers in clinical practice for the direct detection of ROS with PET imaging.

A need remains for improved PET imaging tools to detect ROS.

### SUMMARY OF THE INVENTION

An aspect of the invention provides a radiolabelled compound of formula (I):

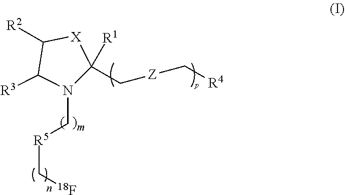

wherein:
X is selected from —O—, —S— or —NR²⁰—;
Z is a double bond or a triple bond;
R¹ is —H or —D;
R² and R³ are linked to form part of an optionally substituted 5- or 6-membered aromatic ring;
R⁴ is selected from —H, optionally substituted alkyl, optionally substituted alkenyl, optionally substituted alkynyl, and optionally substituted aryl;
R⁵ is selected from optionally substituted alkylene, optionally substituted alkenylene, optionally substituted alkynylene and optionally substituted arylene;
R²⁰ is selected from —H, alkyl, alkenyl, alkynyl, acyl and aryl;
n is an integer from 0 to 18;
m is and integer from 0 to 18; and
p is an integer from 0 to 18;
or a pharmaceutically acceptable salt thereof.

A further aspect of the invention provides a reference compound of formula (II):

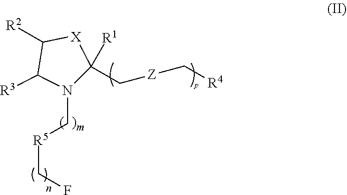

wherein X, Z, R¹, R², R³, R⁴, R⁵, R²⁰, n, m and p are as defined above for the radiolabelled compound of formula (I);
or a pharmaceutically acceptable salt thereof.

Another aspect of the invention provides a precursor compound of formula (III):

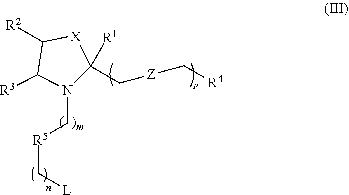

wherein
X, Z, R¹, R², R³, R⁴, R⁵, R²⁰, n, m and p are as defined above for the radiolabelled compound of formula (I); and
L is a leaving group;
or a pharmaceutically acceptable salt thereof.

A further aspect of the invention provides a pharmaceutical composition comprising a radiolabelled compound of formula (I) and a pharmaceutically acceptable carrier.

Another aspect of the invention provides a process for radiolabelling a precursor compound to form the radiolabelled compound of formula (I).

Still a further aspect of the invention provides a process for making a precursor compound or a reference compound, the method comprising the steps of:
(i) reacting a compound of formula (A) with a compound of formula (E) via a cyclization reaction to form a compound of formula (B); and
(ii) reacting the compound of formula (B) with a compound of formula (F) via a nucleophilic addition reaction to form a compound of formula (C); and
(iii) reducing the compound of formula (C) to form a compound of formula (D);
   in accordance with the following reaction scheme:

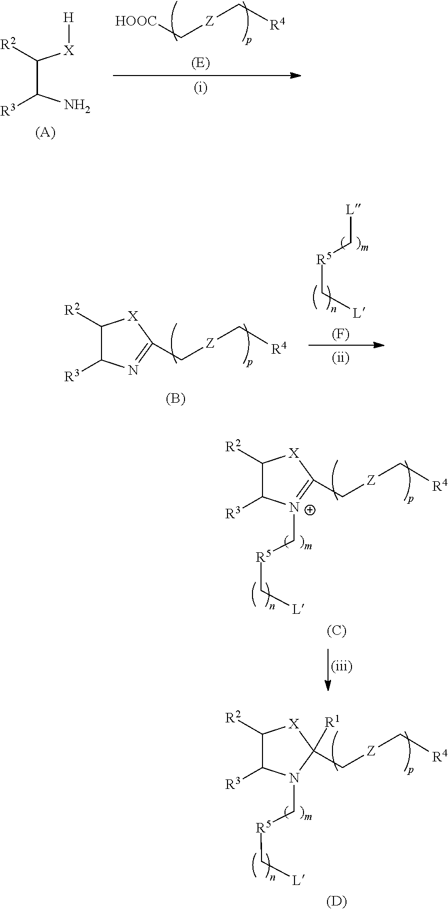

wherein X, Z, R¹, R², R³, R⁴, R⁵, R²⁰, n, m and p are as defined above for the radiolabelled compound of formula (I);

L′ is —F or a leaving group L as defined above for the precursor compound of formula (III); and

L″ is a leaving group.

Still another aspect of the invention provides a radiolabelled compound of formula (I) for use in a diagnostic method practised on the human or animal body using positron emission tomography (PET).

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a proposed mechanism of PET radiotracer ¹⁸F-FM074 for the direct detection of intracellular oxidative stress.

FIG. 2 shows (A) HPLC chromatogram of ¹⁸F-FM074 preparation; (B) co-elution of ¹⁸F-FM074 with its non-radioactive reference compound; (C) HPLC chromatogram of ¹⁸F-FM108 preparation; (D) co-elution of ¹⁸F-FM108 with its non-radioactive reference compound.

FIG. 3 shows (A) the stability of ¹⁸F-FM074 in rat serum at 37° C. in 1 hour determined by radioHPLC; (B) the stability of ¹⁸F-FM108 in rat serum at 37° C. in 30 minutes determined by radioHPLC (30 min above, 5 min below).

FIG. 4 shows the chemoselectivity of (A) [¹⁸F]FM074 and (B) [¹⁸F]FM108.

FIG. 5 shows PET/CT imaging of ¹⁸F-FM074 in mice: a) PET/CT scans of a representative mouse at 1, 5, 20, and 60 min post-injection; b) radioactivity uptake in heart; c) radioactivity uptake in brain; d) radioactivity uptake in kidneys; e) radioactivity uptake in liver. Data represented at SUV mean±SD, n=3.

FIG. 6 shows the biodistribution of (A) ¹⁸F-FM074 and (B) ¹⁸F-FM074-Ox in healthy rats at 1, 5, and 30 min post intravenous injection.

FIG. 7 shows the ejection fraction for control and treated animals before and after treatment in a rat model of Doxorubicin-induced cardiotoxicity.

FIG. 8 shows representative sagittal, coronal, and axial images from co-registered

PET/CT imaging of ¹⁸F-FM074 in Wistar rats, following 7-day exposure to saline (n =4, top) or doxorubicin (n=6, bottom). Legend: M-minipump; LV-left ventricle.

FIG. 9 shows SUVR LV/blood at 3, 10, and 30 min post-injection of ¹⁸F-FM074.

FIG. 10 shows time-activity curves (standard uptake values vs time) of ¹⁸F-FM074 uptake in the left ventricle (LV) and blood pool inside the myocardium in a rat model of Doxorubicin-induced cardiotoxicity.

### DETAILED DESCRIPTION

Aspects of the invention provide or utilise a radiolabelled compound.

Suitably, the radiolabelled compound is a compound of formula (I):

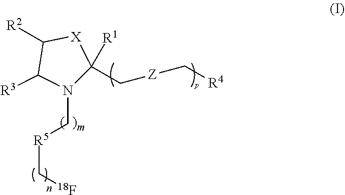

wherein:
X is selected from —O—, —S— or —NR²⁰—;
Z is a double bond or a triple bond;
R¹ is —H or —D;
R² and R³ are linked to form part of an optionally substituted 5- or 6-membered aromatic ring;
R⁴ is selected from —H, optionally substituted alkyl, optionally substituted alkenyl, optionally substituted alkynyl, and optionally substituted aryl;
R⁵ is selected from optionally substituted alkylene, optionally substituted alkenylene, optionally substituted alkynylene and optionally substituted arylene;
R²⁰ is selected from —H, alkyl, alkenyl, alkynyl, acyl and aryl;
n is an integer from 0 to 18;
m is and integer from 0 to 18; and
p is an integer from 0 to 18;
or a pharmaceutically acceptable salt thereof.

In some embodiments, the radiolabelled compound of formula (I) can be used as a radiotracer in positron emission tomography (PET). As shown by the Examples, the compound can have favourable physicochemical properties for unassisted cell membrane permeability and blood-brain-barrier penetration. The radiolabelled compound of formula (I) may be oxidised intracellularly:

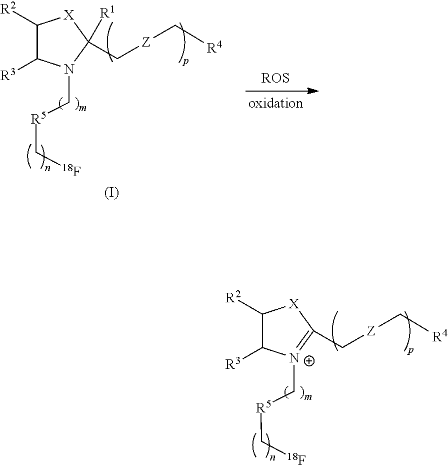

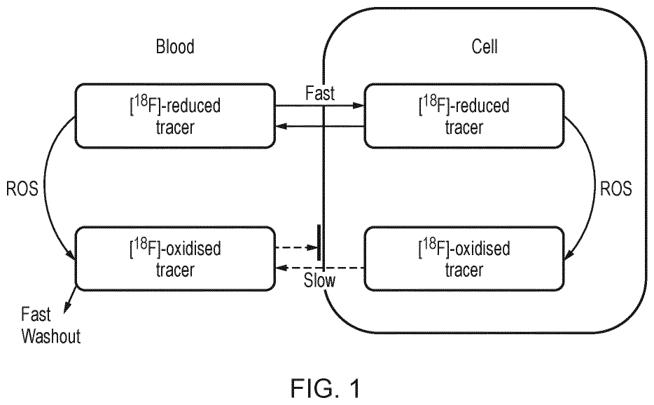

Upon intracellular ROS oxidation, the compound may become cationic and more hydrophilic, which may change its pharmacokinetics, thus increasing its intracellular retention (see FIG. 1).

X is selected from —O—, —S— or —NR²⁰—. Preferably, X is —O— or —S—. More preferably, X is —S—.

Z is a double bond or a triple bond. In an embodiment, Z is a double bond. In another embodiment, Z is a triple bond. When p is zero, Z is not present.

R¹ is —H or —D. In an embodiment, R¹ is —H. In another embodiment, R¹ is —D. Deuterium may increase the oxidative potential of the compound.

R² and R³ are linked to form part of an optionally substituted 5- or 6-membered aromatic ring. It is possible for R² and/or R³ to be a heteroatom, such as nitrogen, oxygen, or sulphur.

In an embodiment, the aromatic ring in the optionally substituted 5- or 6-membered aromatic ring is an optionally substituted 5-membered aromatic ring. Alternatively, the aromatic ring in the optionally substituted 5- or 6-membered aromatic ring is an optionally substituted 6-membered aromatic ring.

In the definition of the optionally substituted 5- or 6-membered aromatic ring, in addition to the substituents listed under the definition of an optionally substituted group below, the substituents may also be joined with the 5- or 6-membered aromatic ring to form fused rings.

In an embodiment, the aromatic ring in the optionally substituted 5- or 6-membered aromatic ring can be an all-carbon aromatic ring, for example benzene; or a heteroaromatic ring, for example pyridine, pyrimidine, pyrazine, pyrrole, imidazole, pyrazole, furan, thiophene, oxazole, isoxazole, or thiazole.

Preferably, the aromatic ring in the optionally substituted 5- or 6-membered aromatic ring is selected from benzene, pyridine, pyrimidine, pyrazine, pyridazine, furan, thiophene and pyrrole.

Preferably, R² and R³ are linked to form part of a 5- or 6-membered aromatic ring selected from:

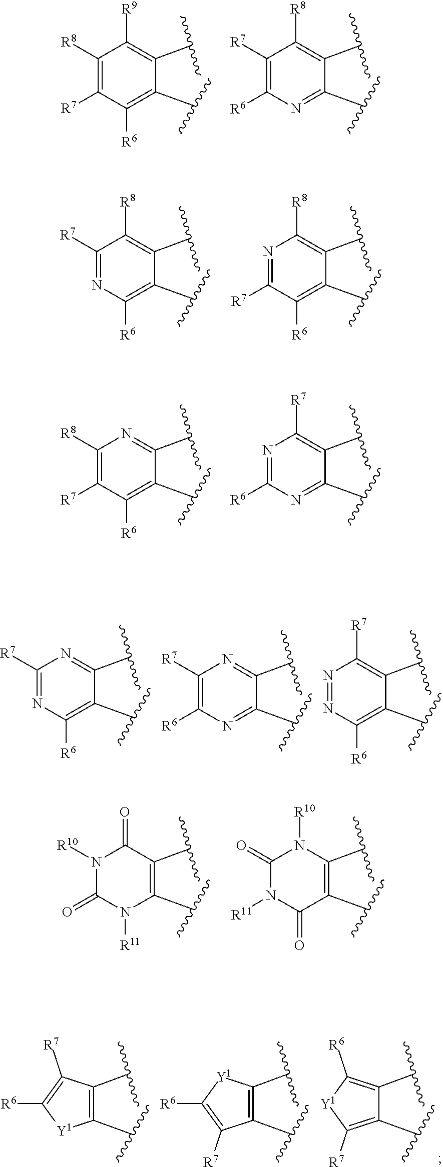

wherein

Y¹ is selected from —O—, —S— and —NR²⁰—;

R⁶, R⁷, R⁸ and R⁹ are independently selected from —H, alkyl, alkenyl, alkynyl, aryl, —CF₃, halogen, —B(OR²⁰)₂, —OR²⁰, —NR²⁰₂, —SR²⁰, —SiR²⁰³, —503, —503R²⁰, —SO₂NR²⁰², —S(O)R²⁰, —C(O)R²⁰, —C(O)NR²⁰₂, —CO2R²⁰, —NO₂, and -CN;

R¹⁰ and R¹¹ are independently selected from —H, alkyl, alkenyl alkynyl, acyl, and aryl; and R²⁰ is as defined above.

Preferably, R⁶, R⁷, R⁸ and R⁹ are independently selected from —H and alkyl. More preferably, each of R⁶, R⁷, R⁸ and R⁹ is —H.

Preferably, R² and R³ are linked to form

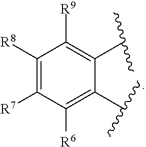

More preferably, R² and R³ are linked to form part of a benzene ring.

R⁴ is selected from —H, optionally substituted alkyl, optionally substituted alkenyl, optionally substituted alkynyl, and optionally substituted aryl.

In an embodiment, R⁴ is —H.

Suitably, R⁴ may be optionally substituted alkyl. The alkyl group in the optionally substituted alkyl may be a C₁₋₂₀ alkyl group, or a C_{1_}12 alkyl group, such as a C_{1_}8 alkyl group, for example a C₁₋₆ alkyl group, or a C₁₋₄ alkyl group, for example methyl or ethyl.

R⁴ may be optionally substituted alkenyl. The alkenyl group in the optionally substituted alkenyl may be a C₂₋₂₀ alkenyl group, or a C₂₋₁₂ alkenyl group, such as a C₂₋₈ alkenyl group, for example a C₂₋₆ alkenyl group, or a C₂₋₄ alkenyl group, for example vinyl (—CH═CH₂).

R⁴ may be optionally substituted alkynyl. The alkynyl group in the optionally substituted alkynyl may be a C₂₋₂₀ alkynyl group, or a C₂₋₁₂ alkynyl group, such as a C₂₋₈ alkynyl group, for example a C₂₋₆ alkynyl group, or a C₂₋₄ alkynyl group, for example ethynyl (—CH═CH).

Preferably, R⁴ may be optionally substituted aryl. The aryl group in the optionally substituted aryl can be an all-carbon aromatic moiety, for example phenyl (derived from benzene) or naphthyl (derived from naththalene); or a heteroaryl moiety, for example pyridinyl (or pyridyl, derived from pyridine), pyrimidinyl (derived from pyrimidine), pyrazinyl (derived from pyrazine), pyrrolyl (derived from pyrrole), imidazolyl (derived from imidazole), pyrazolyl (derived from pyrazole), furyl (derived from furan), thiophenyl (derived from thiophene), oxazolyl (derived from oxazole), isoxazolyl (derived from isoxazole), or thiazolyl (derived from thiazole).

When R⁴ is optionally substituted aryl, the aryl group in the optionally substituted aryl may suitably be a 5- or 6-membered aromatic moiety, for example phenyl, pyridinyl, pyrimidinyl, pyrazinyl, pyrrolyl, imidazolyl, pyrazolyl, furyl, thiophenyl, oxazolyl, isoxazolyl or thiazolyl. Preferably, the optionally substituted aryl is optionally substituted phenyl, optionally substituted pyridinyl, optionally substituted pyrrolyl, optionally substituted furyl, thiophenyl, or optionally substituted thiophenyl. More preferably, R⁴ is an optionally substituted phenyl group.

In a preferred embodiment, R⁴ is a 5- or 6-membered aromatic moiety selected from:

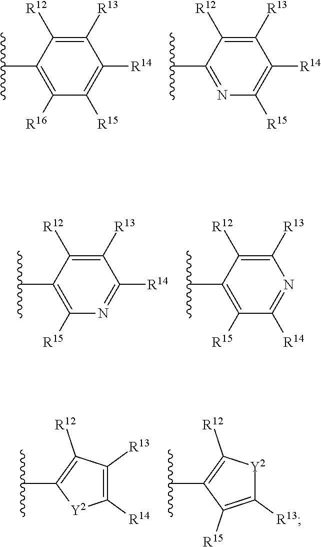

wherein

Y₂ is selected from —O—, —S— and —NR²⁰—;

R¹², R¹³, R¹⁴, R′5 and R¹⁶ are independently selected from —H, alkyl, alkenyl, alkynyl, aryl, —CF₃, halogen, —B(OR²⁰)₂, —OR²⁰, —NR²⁰₂, —SR²⁰, —SiR²⁰₃, —SO₃, —SO₃R²⁰, —SO₂NR²⁰₂, —S(O)R²⁰, —C(O)R²⁰, —C(O)NR²⁰₂, —CO₂R²⁰, —NO₂, and —CN; and

R²⁰ is as defined above.

Preferably, R¹², R¹³, R¹⁴, R¹⁵ and R¹⁶ are independently selected from —H and alkyl. More preferably, each of R¹², R¹³, R¹⁴, R¹⁵ and R¹⁶ is —H.

Preferably, R⁴ is

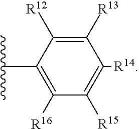

More preferably, R⁴ is phenyl.

R⁵ is selected from optionally substituted alkylene, optionally substituted alkenylene, optionally substituted alkynylene and optionally substituted arylene.

In an embodiment, R⁵ is optionally substituted alkylene. Preferably, R⁵ is —(CH₂)_{q}— wherein q is an integer from 1 to 20. In an embodiment, q is 1, 2, 3, 4, 5, 6, 7, 8, 9, 10, 11, 12, 13, 14, 15, 16, 17, 18, 19 or 20. R⁵ may, for example, be methylene, ethylene, propylene or butylene.

In a preferred embodiment, n and m are both zero, and R⁵ is —(CH₂)_{q}— wherein q is an integer from 1 to 20, for example from 1 to 6. In an embodiment, q is 0, 1, 2, 3, 4, 5, 6, 7, 8, 9, 10, 11, 12, 13, 14, 15, 16, 17, 18, 19 or 20. Preferably, q is 0, 1, 2, 3, 4, 5 or 6. More preferably, n and m are both zero, and R⁵ is propylene (q is 3).

In another embodiment, R⁵ is optionally substituted arylene. The arylene group in the optionally substituted aryl may suitably be a 5- or 6-membered aromatic moiety, for example phenylene, pyridinylene, pyrimidinylene, pyrazinylene, pyrrolylene, imidazolylene, pyrazolylene, furylene, thiophenylene, oxazolylene, isoxazolylene or thiazolylene. Preferably, the optionally substituted arylene is optionally substituted phenylene.

Throughout this specification, R²⁰ is selected from —H, alkyl, alkenyl, alkynyl, aryl and acyl; for example —H, C₁₋₂₀alkyl, C₂₋₂₀alkenyl, C₂₋₂₀alkynyl, C₃₋₂₀aryl and acyl. Preferably, R²⁰ is —H or C₁₋₂₀alkyl, such as C₁₋₆alkyl.

n is an integer from 0 to 18.

In an embodiment, n is 0, 1, 2, 3, 4, 5, 6, 7, 8, 9, 10, 11, 12, 13, 14, 15, 16, 17 or 18. Preferably, n is 0, 1, 2, 3, 4, 5 or 6. More preferably, n is 0 or 1. Most preferably, n is 0.

m is and integer from 0 to 18.

In an embodiment, m is 0, 1, 2, 3, 4, 5, 6, 7, 8, 9, 10, 11, 12, 13, 14, 15, 16, 17 or 18. Preferably, m is 0, 1, 2, 3, 4, 5 or 6. More preferably, m is 0 or 1. Most preferably, m is 0.

p is an integer from 0 to 18.

In an embodiment, p is 0, 1, 2, 3, 4, 5, 6, 7, 8, 9, 10, 11, 12, 13, 14, 15, 16, 17 or 18. In an embodiment, p is 0 or 1. Preferably, p is 0.

In an embodiment, the compound of formula (I) is a phenyl benzothiazole.

In an embodiment, the compound of formula (I) is a compound of formula:

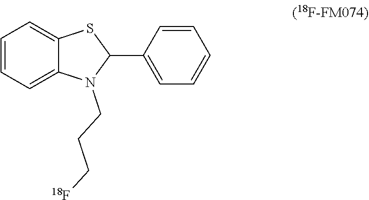 [SMILES: [18F]CCCN1c2ccccc2SC1c1ccccc1]

This compound is [¹⁸F]3-(3-fluoropropyl)-2-phenyl-2,3-dihydrobenzo[d]thiazole. It is a phenyl benzothiazole, and is interchangeably referred to as ¹⁸F-FM074, [¹⁸F]FM074, or ¹⁸F-ROS-PROBE throughout this specification.

The compound of formula (I) may be present in the form of a pharmaceutically acceptable salt.

A further aspect of the invention provides a reference compound of formula (II):

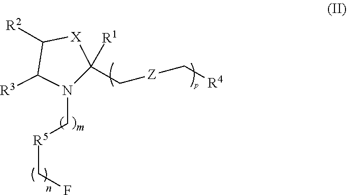

wherein X, Z, R¹, R², R³, R⁴, R⁵, R²⁰, n, m and p are as defined above for the radiolabelled compound of formula (I);
or a pharmaceutically acceptable salt thereof.

The reference compound of formula (II) can function as a reference compound for the radiolabelled compound of formula (I), the reference compound having an F atom in place of the ¹⁸F atom in formula (I).

Another aspect of the invention provides a precursor compound of formula (III):

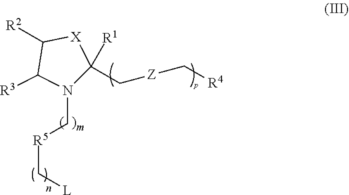

wherein
X, Z, R¹, R², R³, R⁴, R⁵, R²⁰, n, m and p are as defined above for the radiolabelled compound of formula (I); and
L is a leaving group;
or a pharmaceutically acceptable salt thereof.

The precursor compound of formula (III) can function as a precursor compound for the radiolabelled compound of formula (I), the precursor compound having a leaving group in place of the ¹⁸F atom in formula (I).

Preferably, L is selected from —I, —Br, —Cl, and sulfonate esters such as for example triflate (—OTf), mesylate (—OMs), and tosylate (—OTs). More preferably, L is selected from —I, —Br, —OTf, —OMs and —OTs. Most preferably, L is —I.

A further aspect of the invention provides a pharmaceutical composition comprising a radiolabelled compound of formula (I) as defined above and a pharmaceutically acceptable carrier.

Another aspect of the invention provides a process for radiolabelling a precursor compound to form the radiolabelled compound of formula (I).

The radiolabelled compound of formula (I) contains the ¹⁸F radioisotope. ¹⁸F-fluoride is a commonly used PET radioisotope, due to the mainstream use of ¹⁸F-FDG (2-deoxy-2-[fluorine-18]fluoro-D-glucose) scans. ¹⁸F has a relatively short half-life (t_{1/2}=109 min). The radiolabelling process of the invention can provide a ‘late stage’ ¹⁸F-labelling strategy, where the radioactive isotope is only added in the final process step before use. Furthermore, as can be seen from the Examples, the radiolabelling step of the invention achieves very high radiochemical yields and molar activity. Compared to other known PET radiotracers with (much) lower radiolabelling yields, this means that smaller amounts of ¹⁸F are required to achieve the same amount of radiotracer, resulting in less exposure for radiotechnicians preparing the radiotracer for use in PET imaging. In addition, it means that the radiolabelling step can readily be automated for large scale, using existing radiosynthesiser methodology.

Preferably, the precursor compound in the radiolabelling process may be the precursor compound of formula (III) defined above.

Suitably, the process comprises the step of reacting the precursor compound of formula (III) with nucleophilic fluoride-18 to form the radiolabelled compound of formula (I), in accordance with the following reaction scheme:

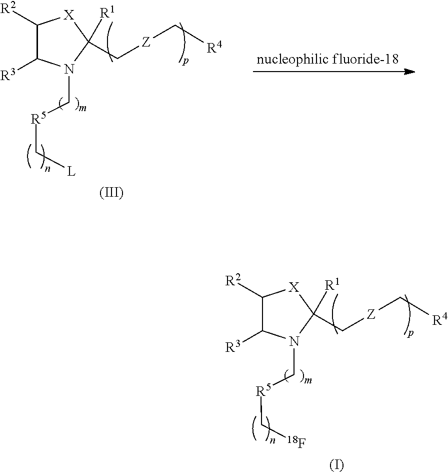

wherein X, Z, R¹, R², R³, R⁴, R⁵, R²⁰, n, m and p are as defined above; and L is as defined above.

Suitably, the nucleophilic fluoride-18 may for example be provided in the form of a compound selected from K¹⁸F, Cs¹⁸F, tBu₄N¹⁸F and Et₄N¹⁸F.

Still a further aspect of the invention provides a process for making a precursor compound or a reference compound, the method comprising the steps of:
(i) reacting a compound of formula (A) with a compound of formula (E) via a cyclization reaction to form a compound of formula (B); and
(ii) reacting the compound of formula (B) with a compound of formula (F) via a nucleophilic addition reaction to form a compound of formula (C); and
(iii) reducing the compound of formula (C) to form a compound of formula (D);
   in accordance with the following reaction scheme:

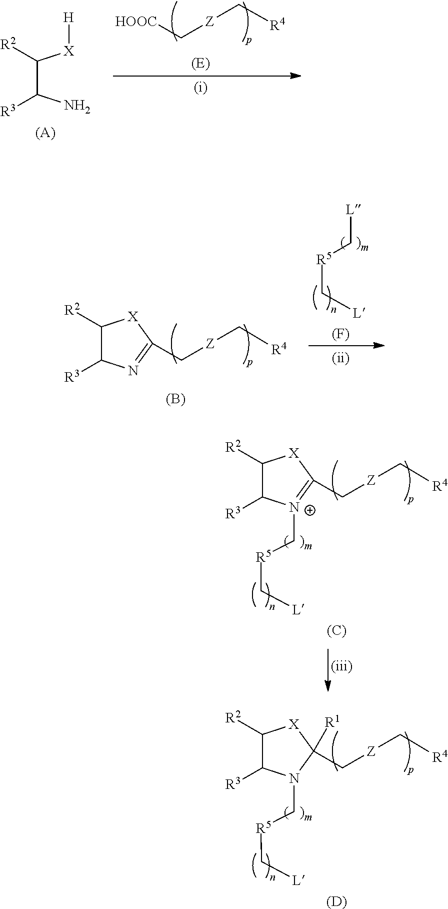

wherein X, Z, R¹, R², R³, R⁴, R⁵, R²⁰, n, m and p are as defined above for the radiolabelled compound of formula (I);
L′ is —F or a leaving group L as defined above for the precursor compound of formula (III); and
L″ is a leaving group.

The process may be a process for making a precursor compound or a reference compound for the radiolabelled compound of formula (I),

In an embodiment, the process is a process for making the reference compound of formula (II) defined above. In that embodiment, the compound of formula (D) is the reference compound of formula (II), and L′ is —F.

In another embodiment, the process is a process for making the precursor compound of formula (III) defined above. In that embodiment, the compound of formula (D) is the precursor compound of formula (III), and L′ is a leaving group L as defined above. Preferably, the leaving group L may be selected from —I and —Br.

Suitably, L″ may be a better leaving group than L. For example, L″ may be a more reactive leaving group than L.

Preferably, leaving group L″ is a sulfonate ester. The sulfonate ester can for example be selected from triflate (—OTf), mesylate (—OMs), and tosylate (—OTs); more preferably triflate (—OTf).

The precursor compound for radiolabelling provided by the invention can be easily prepared in only a few synthetic steps. Thus, the production of the precursor compound for clinical use is easy and economical.

In an embodiment of the process for making a precursor compound or a reference compound, the process further comprises the step of converting a compound of formula (G) to the compound of formula (F), in accordance with the following reaction scheme:

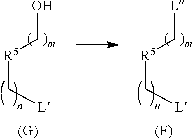

wherein R⁵, n, m, L′ and L″ are as defined above.

Still another aspect of the invention provides a radiolabelled compound of formula (I) defined above for use in a diagnostic method practised on the human or animal body using positron emission tomography (PET).

Preferably, the diagnostic method is a method of detecting reactive oxygen species in the human or animal body.

Suitably, the diagnostic method may be a method of diagnosing a condition which is caused by and/or exacerbated by elevated ROS production.

The condition which is caused by and/or exacerbated by elevated ROS production may, for example, be a neurodegenerative disease, for example Alzheimer's disease or Parkinson's disease. Alternatively, the condition which is caused by and/or exacerbated by elevated ROS production may for example be atherosclerotic plaques. Detection of vulnerable atherosclerotic plaques could prevent plaque rupture causing myocardial infarction.

Suitably, the diagnostic method may be a method of monitoring the therapeutic efficacy of a therapy, for example cancer chemotherapy or radiotherapy, or antioxidant therapy.

### Definitions

It is to be understood that the wavy line in any chemical structures or moieties represented herein, such as shown below, indicates the point of attachment of that structure or moiety.

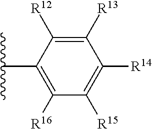

The term “hydrogen” or “hydrogen atom” as used herein refers to a —H moiety.

The term “halo”, “halogen” or “halogen atom” as used herein refers to a —F, —Cl, —Br or —I moiety.

The term “hydroxy” or “hydroxyl” as used herein refers to an —OH moiety.

10 The prefix “Cₓ”“C_{x-y}” denotes the number of carbon atoms, or range of number of carbon atoms present in that group. Thus, the term “C₁₋₁₂ alkyl” refers to an alkyl group having from 1 to 12 carbon atoms.

The term “alkyl” refers to a monovalent moiety obtained by removing a hydrogen atom from a carbon atom of a saturated hydrocarbon compound, for example having from 1 to 20 carbon atoms, which may be linear, branched, or cyclic. Thus the term “alkyl” includes the subclass cycloalkyl below. Examples of alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉) and decyl (C₁₀). Examples of linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇). Examples of branched alkyl groups include, but are not limited to, iso-propyl (C₃), iso-butyl (C₄), sec-butyl (C₄), tert-butyl (C₄), iso-pentyl (C₅), and neo-pentyl (C₅).

The term “cycloalkyl” refers a monovalent moiety obtained by removing a hydrogen atom from a carbon atom of a cyclic saturated hydrocarbon compound, for example having from 3 to 20 carbon atoms. “Cycloalkyl” includes monocyclic and polycyclic rings including bicyclic rings. Examples of monocyclic cycloalkyl groups include, but are not limited to, cyclopropyl (C₃), cyclobutyl (C₄), cyclopentyl (C₅), cyclohexyl (C₆), cycloheptyl (C₇) and methylcyclopropyl (C₄). Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a bicyclic cycloalkyl may be selected from saturated, unsaturated and aromatic rings. Examples of saturated polycyclic hydrocarbon compounds include, but are not limited to, thujane (C₁₀), carane (C₁₀), pinane (C₁₀), bornane (C₁₀), norcarane (C₇), norpinane (C₇), norbornane (C₇), adamantane (C₁₀) and decalin (C₁₀).

The term “alkenyl” refers to a monovalent hydrocarbon moiety obtained by removing a hydrogen atom from a carbon atom of a (partially) unsaturated hydrocarbon compound having one or more carbon-carbon double bonds, and for example having from 2 to 20 carbon atoms, which may be linear, branched, or cyclic. Thus the term “alkenyl” includes the subclass cycloalkenyl below. Examples of alkenyl groups include, but are not limited to ethenyl (vinyl, —CH═CH₂), 1-propenyl (—CH═CH—CH₃) and 2-propenyl (allyl, —CH-CH═CH₂).

The term “cycloalkenyl” refers to a monovalent cyclic hydrocarbon moiety obtained by removing a hydrogen atom from a carbon atom of a cyclic (partially) unsaturated hydrocarbon compound containing one or more carbon-carbon double bonds, and for example having from 3 to 20 carbon atoms. “Cycloalkenyl” includes monocyclic and polycyclic rings including bicyclic rings. Examples of unsaturated monocyclic hydrocarbon compounds include, but are not limited to cyclopropene (C₃), cyclobutene (C₄), cyclopentene (C₅), cyclohexene (C₆), methylcyclopropene (C₄) and dimethylcyclopropene (C₅). Examples of unsaturated polycyclic hydrocarbon compounds include, but are not limited to camphene (C₁₀), limonene (C₁₀) and pinene (C₁₀)

The term “alkynyl” refers to a monovalent hydrocarbon moiety obtained by removing a hydrogen atom from a carbon atom of a (partially) unsaturated hydrocarbon compound having one or more carbon-carbon triple bonds, and for example having from 2 to 20 carbon atoms, which may be linear, branched, or cyclic. Thus the term “alkynyl” includes the subclass cycloalkynyl below. Examples of alkynyl groups include, but are not limited to, ethynyl (ethinyl, —C≡CH) and 2-propynyl (propargyl, —CH₂≡CECH).

The term “cycloalkynyl” refers to a monovalent cyclic hydrocarbon moiety obtained by removing a hydrogen atom from a carbon atom of a cyclic (partially) unsaturated hydrocarbon compound containing one or more carbon-carbon triple bonds, and for example having from 2 to 20 carbon atoms. “Cycloalkynyl” includes monocyclic and polycyclic rings including bicyclic rings.

The term “aryl” refers to a monovalent moiety obtained by removing a hydrogen atom from a ring atom of an aromatic compound, which moiety may for example be a monocyclic or bicyclic group. The aromatic compound which the aryl group is derived from may contain an all-carbon ring structure or may be a heteroaromatic compound containing one or more heteroatoms in the ring structure. Thus the term “aryl” includes the subclass heteroaryl below. An aryl group with an all-carbon ring structure may for example have from 3 to 20 carbon atoms. Examples of aryl groups include, but are not limited to, phenyl (derived from benzene) and naphthyl (derived from naphthalene).

The term “heteroaryl” refers to a monovalent moiety obtained by removing a hydrogen atom from a ring atom of a heteroaromatic compound, which moiety may for example be a monocyclic or bicyclic group. The heteroaryl moiety may for example contain one or more N, O, S or P atoms, and may for example contain from 1 to 20 carbon atoms. Examples of heteroaryl groups include, but are not limited to, pyridinyl (or pyridyl, derived from pyridine), pyrimidinyl (derived from pyrimidine), pyrazinyl (derived from pyrazine), pyrrolyl (derived from pyrrole), imidazolyl (derived from imidazole), pyrazolyl (derived from pyrazole), furyl (derived from furan), thiophenyl (derived from thiophene), oxazolyl (derived from oxazole), isoxazolyl (derived from isoxazole), and thiazolyl (derived from thiazole).

The term “heterocyclyl” refers to a monovalent moiety obtained by removing a hydrogen atom from a ring atom of a heterocyclic compound, which moiety may for example be a monocyclic or bicyclic group. The heterocyclyl group may for example contain one or more N, O, S or P atoms, and may for example contain from 1 to 20 carbon atoms.

The term “alkoxy” or “alkoxyl” refers to an alkyl-oxy group, where the alkyl group is as defined above. Examples of alkoxy groups include, but are not limited to -OMe (methoxy), -OEt (ethoxy), —O(ⁿPr) (n-propoxy), —O(ⁱPr) (isopropoxy), —O(ⁿBu) (n-butoxy), —O(^{s}Bu) (sec-butoxy), —O(ⁱBu) (isobutoxy), and -O(tBu) (tert-butoxy).

The term “acyl” refers to a group represented by the general formula —C(O)-hydrocarbyl, such as —C(O)-alkyl.

The term “alkylene” refers to a divalent hydrocarbon moiety obtained by removing two hydrogen atoms, either both from the same carbon atom, or one from each of two different carbon atoms, of a saturated hydrocarbon compound, for example having from 1 to 20 carbon atoms, which may be linear, branched, or cyclic. Thus the term “alkylene” includes the subclass cycloalkylene below. Examples of linear alkylene groups include, but are not limited to, —CH₂— (methylene), —CH₂CH₂— (ethylene), —CH₂CH₂CH₂- (propylene), and —CH₂CH₂CH₂CH₂— (butylene). Examples of branched alkylene groups include, but are not limited to, —CH(CH₃)—, —CH(CH₃)CH₂—, and —CH(CH₃)CH₂CH₂—.

The term “cycloalkylene” refers to a divalent moiety obtained by removing two hydrogen atoms, either both from the same carbon atom, or one from each of two different carbon atoms, of a cyclic saturated hydrocarbon compound, for example having from 3 to 20 carbon atoms. “Cycloalkylene” includes monocyclic and polycyclic rings including bicyclic rings. Examples of cyclic alkylene groups include, but are not limited to, cyclopentylene (e.g. cyclopent-1,3-ylene) and cyclohexylene (e.g. cyclohex-1,4-ylene).

The term “alkenylene” refers to a divalent hydrocarbon moiety obtained by removing two hydrogen atoms, either both from the same carbon atom, or one from each of two different carbon atoms, of a (partially) unsaturated hydrocarbon compound having one or more carbon-carbon double bonds, and for example having from 2 to 20 carbon atoms, which may be linear, branched, or cyclic. Thus the term “alkenylene” includes the subclass cycloalkenylene below. Examples of linear alkenylene groups include, but are not limited to, —CH═CH— (vinylene), —CH═CHCH₂—, —CH₂—CH═CH₂—, and —CH═CHCH₂CH₂—. Examples of branched alkenylene groups include, but are not limited to, —C(CH₃)=CH—, —C(CH₃)=CHCH₂— and —CH═CHCH(CH₃)—.

The term “cycloalkenylene” refers to a divalent moiety obtained by removing two hydrogen atoms, either both from the same carbon atom, or one from each of two different carbon atoms, of a cyclic (partially) unsaturated hydrocarbon compound having one or more carbon-carbon double bonds, and for example having from 3 to 20 carbon atoms. “Cycloalkenylene” includes monocyclic and polycyclic rings including bicyclic rings. Examples of cycloalkenylene groups include, but are not limited to, cyclopentenylene (e.g. 4-cyclopenten-1,3-ylene) and cyclohexenylene (e.g. 2-cyclohexen-1,4-ylene; 3-cyclohexen-1,2-ylene; 2,5-cyclohexadien-1,4-ylene).

The term “alkynylene” refers to a divalent moiety obtained by removing two hydrogen atoms, either both from the same carbon atom, or one from each of two different carbon atoms, of a (partially) unsaturated hydrocarbon compound having one or more carbon-carbon triple bonds, and for example having from 2 to 20 carbon atoms, which may be linear, branched, or cyclic. Thus the term “alkenylene” includes the subclass cycloalkenylene below.

The term “cycloalkynylene” refers to a divalent moiety obtained by removing two hydrogen atoms, either both from the same carbon atom, or one from each of two different carbon atoms, of a cyclic (partially) unsaturated hydrocarbon compound containing one or more carbon-carbon triple bonds, and for example having from 3 to 20 carbon atoms. “Cycloalkynylene” includes monocyclic and polycyclic rings including bicyclic rings.

The term “arylene” refers to a divalent moiety obtained by removing two hydrogen atoms, either both from the same carbon atom, or one from each of two different carbon atoms, of an aromatic compound, which moiety may for example be a monocyclic or bicyclic group. The aromatic compound which the arylene group is derived from may contain an all-carbon ring structure or may be a heteroaromatic compound containing heteroatoms in the ring structure. Thus the term “arylene” includes the subclass heteroarylene below. An arylene group with an all-carbon ring structure may for example have from 3 to 20 carbon atoms.

The term “heteroarylene” refers to a divalent moiety obtained by removing two hydrogen atoms, either both from the same ring atom, or one from each of two different ring atoms, of a heteroaromatic compound, which moiety may for example be a monocyclic or bicyclic group. The heteroarylene moiety may for example contain one or more N, 0, S or P atoms, and may for example contain from 1 to 20 carbon atoms.

The term “substituent” refers to a chemical moiety, which is covalently attached to, or if appropriate, fused to, a parent group.

The phrase “optionally substituted” refers to a parent group which may be unsubstituted or which may be substituted with one or more, for example one or two, substituents. The substituents on an “optionally substituted” group may for example be selected from alkyl, alkenyl, alkynyl, aryl, heteroaryl and heterocyclyl groups; carboxylic acids and carboxylate ions; carboxylate esters; carbamates; alkoxy groups; ketone and aldehyde groups; amine and amide groups; —OH; —CN; —NO₂; and halogens.

The term “protecting group” as used herein refers to a group capable of protecting a functional group (e.g. a heteroatom such as an oxygen atom), which protecting group may, subsequent to the reaction for which protection is employed, be removed without disturbing the remainder of the molecule. Protecting groups are well known and listed in standard texts such as Kocienski P. J., Protecting Groups, 3rd ed., Georg Thieme Verlag, New York, 2005; and Greene T. W., Wuts P. G. M., Protective Groups In Organic Synthesis, 3rd ed., John Wiley & Sons, New York, 1998.

Certain compounds may exist in one or more particular geometric, enantiomeric, diastereomeric, tautomeric, or conformational forms. Unless otherwise specified, a reference to a particular compound includes all such isomeric forms, including (wholly or partially) racemic and other mixtures thereof. Methods for the preparation and separation of such isomeric forms are known in the art.

Pharmaceutically acceptable salt forms include pharmaceutically acceptable acidic/anionic or basic/cationic salts.

Examples of pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Examples of pharmaceutically acceptable basic/cationic salts include sodium, potassium, calcium, magnesium, diethanolamine, N-methyl-D-glucamine, L-lysine, L-arginine, ammonium, ethanolamine, piperazine and triethanolamine salts.

If the compound is anionic, or has a functional group which may be anionic, then a salt may be formed with a suitable cation. Examples of suitable inorganic cations include alkali metal ions, such as Na⁺ and K⁺, alkaline earth cations, such as Ca²⁺ and Mg²⁺, and other cations such as A³⁺. Examples of suitable organic cations include ammonium ion (i.e., NH₄⁺) and substituted ammonium ions (e.g. NH₃R⁺, NH₂R²⁺, NHR²⁺, NR⁴⁺, where R is an alkyl group).

If the compound is cationic, or has a functional group which may be cationic, then a salt may be formed with a suitable anion. Examples of suitable inorganic anions include those derived from the following inorganic acids: hydrochloric, hydrobromic, hydroiodic, sulfuric, sulfurous, nitric, nitrous, phosphoric, and phosphorous. Examples of suitable organic anions include those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric.

If the compound has both a cationic functional group, or a functional group that can become cationic, and an anionic functional group, or a functional group that can become anionic, then the compound may be present as a zwitterion.

The following non-limiting examples are provided by way of illustration only.

### EXAMPLES

Abbreviations

ROS stands for reactive oxygen species.

K222 stands for Kryptofix 222.

¹⁸F-FM074 or [¹⁸F]FM074 stands for [¹⁸F]-(3-fluoropropyl)-2-phenyl-2,3-dihydrobenzo[d]thiazole. This compound is also referred to as ¹⁸F-ROS-PROBE.

¹⁸F-FM074-Ox or [¹⁸F]FM074-Ox stands for [¹⁸F]-(3-fluoropropyl)-2-phenylbenzo[d]thiazol-3-ium chloride or the cation thereof as context may require. This compound is also referred to as ¹⁸F-ROS-PROBE-Ox.

¹⁸F-FM108 or [¹⁸F]FM108 stands for [¹⁸F]3-(3-fluoropropyl)-2-(4-methoxyphenyl)-2,3-dihydrobenzo[d]thiazole

¹⁸F-FM108-Ox or [¹⁸F]FM108-Ox stands for [¹⁸F]3-(3-fluoropropyl)-2-(4-methoxyphenyl)-2,3-dihydrobenzo[d]thiazole-3-ium chloride or the cation thereof as context may require.

1. Synthetic Chemistry

We have synthesized a library of ¹⁸F-labelled molecular probes basing on benzothiazolines for the in vivo visualisation of ROS by PET, as shown in Scheme 1 below. Compounds with general structure 1 are converted into structure 2 upon oxidation.

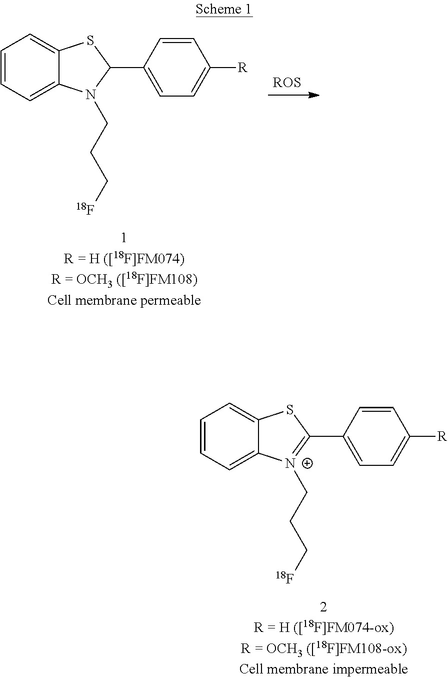

General Synthetic Methods

(a) Synthesis of 2-arylbenzothiazoles

A mixture of aryl carboxylic acid (12.3 mmol, 1.4 equiv.), 2-aminothiophenol (9.5 mmol, 1.0 equiv.) and polyphosphoric acid (3× acid weight) was heated with stirring at 150° C. overnight. Upon cooling, the resulting mixture was neutralised with 7% NH₄OH (25 mL), and stirred for 2 hrs. The green solid was filtered and thoroughly rinsed with 7% NH₄OH solution. The precipitate was recrystallized from a mixture of diethyl ether and hexane to give the desired compound.

(b) Synthesis of 3-halo-propyl trifluoromethanesulfonates

To a solution of trifluoromethanesulfonic anhydride (30 mmol) in anhydrous DCM (15 mL) was added a solution of 3-iodo-1-propanol or 3-fluoro-1-propanol (30 mmol) and anhydrous pyridine (30 mmol) in anhydrous DCM (20 mL) dropwise at 0° C. under nitrogen. A white precipitate of pyridinium salt was formed upon addition. The reaction mixture was stirred at 0° C. for 30 min before quenching with deionised water (20 mL). The organic layer was separated and washed with deionised water (50 mL×2), brine (50 mL), dried over MgSO₄, filtered and concentrated in vacuum to give a light brown liquid (43-63% yield). Full conversion was observed by a new single spot on the TLC plate (EtOAc/hexane 1:1) and the resulting compound was used for next reaction without any further purification.

(c) N-alkylation of 2-arylbenzothiazoles

The 3-halo-propyl trifluoromethanesulfonate (2.5 mmol, 5.0 equiv.) was added to a suspension of NaHCO3 (2.5 mmol, 5.0 equiv.) and the 2-arylbenzothiazone (0.5 mmol, 1.0 equiv.) in nitrobenzene (10 mL). The reaction mixture was stirred at room temperature for 24 hours. The nitrobenzene was removed by a short flash column on silica. The reaction mixture was loaded directly onto the column and eluted with DCM until all nitrobenzene was removed. The crude product on the column was then eluted with DCM/MeOH (9:1). The solvents were removed in vacuum. The quaternary ammonium trifluoromethanesulfonate salts were subjected to reduction without any further purification.

(d) Reduction of Quaternary Ammonium Trifluoromethanesulfonate Salts

The quaternary ammonium salt synthesised in method (c) (0.22 mmol) was dissolved in THF (5 mL) and MeOH (10 mL) in a round-bottom flask covered in aluminium foil. NaBH₄ (0.22 mmol) in methanol (1 mL) was added dropwise to the above brown solution. The resulting solution was stirred for 20 minutes during which time the solution turned colourless. The solvents were removed in vacuum. The resulting solid was re-dissolved in DCM (20 mL) and washed with water (10 mL). The water layer was extracted with additional DCM (2×10 mL). The combined organic phase was washed with brine and dried over anhydrous MgSO₄. The solvent was removed under reduced pressure and the crude material was purified by flash column chromatography using a gradient of 0-30% ethyl acetate in hexane.

HPLC Analysis Method

HPLC analysis was performed using an Agilent column (Eclipse XDB-C18, 4.6×150 mm, 5 μm) using AcCN (0.1% TFA) and water (0.1% TFA) as the mobile phase, at a flow rate of 1 mL/min. The following gradient was used: 5% AcCN for 3 min, 5% to 95% AcCN in 10 min, 95% AcCN for 7 min.

Synthesis of Precursors and Reference Compounds

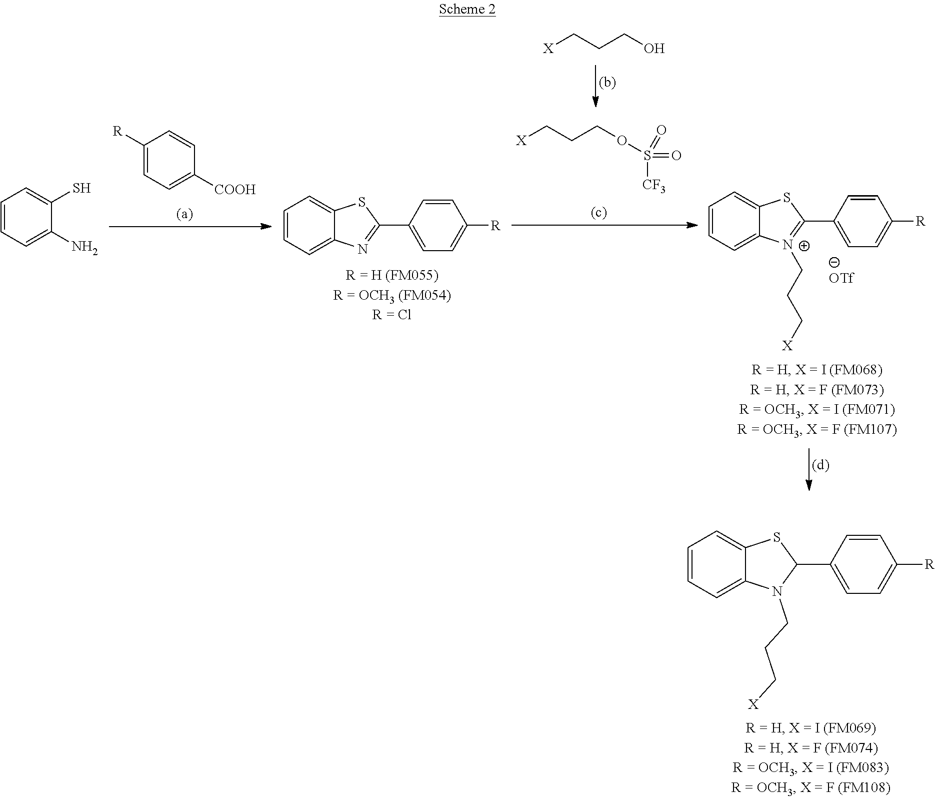

Scheme 2 above shows the preparation of non-radioactive reference compounds of the ¹⁸F-labelled radiotracers (i.e. not containing a radio isotope), and iodinated precursors of the ¹⁸F-labelled radiotracers for radiofluorination, from commercially available starting materials.

2-Phenylbenzo[d]thiazole (FM055)

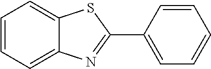 [SMILES: c1ccc(-c2nc3ccccc3s2)cc1]

The title compound was synthesised using method (a) above and isolated in 67% yield.

¹H NMR (400 MHz, DMSO-d₆) δ: 8.19-8.05 (4H, m, Ar—H), 7.63-7.53 (4H, m, Ar—H), 7.48 (1H, ddd, J=8.3, 7.2, 1.2 Hz, Ar—H).

¹³C NMR (101 MHz, DMSO-d₆) δ: 167.25 (qC), 153.50 (qC), 134.40 (qC), 132.79 (qC), 131.41 (CH), 129.39 (CH), 127.16 (CH), 126.65 (CH), 125.53 (CH), 122.86 (CH), 122.36 (CH).

3-(3-Iodopropyl)-2-phenylbenzo[d]thiazol-3-ium trifluoromethanesulfonate (FM068)

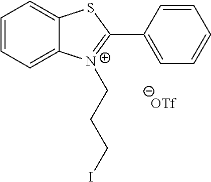 [SMILES: ICCC[n+]1c(-c2ccccc2)sc2ccccc21]

The title compound was synthesised using method (c) above and isolated in 32% yield (crude).

¹H NMR (400 MHz, DMSO-d₆) δ 8.58 (1H, dd, J=8.2, 1.2 Hz, Ar—H), 8.51 (1H, d, J=8.5 Hz, Ar—H), 8.03 (1H, ddd, J=8.5, 7.3, 1.3 Hz, Ar—H), 7.98-7.74 (5H, m, Ar—H), 4.75 (t, 2H, CH₂), 3.32-3.22 (2H, m, CH₂), 2.42-2.31 (2H, m, CH₂).

3-(3-Fluoropropyl)-2-phenylbenzo[d]thiazol-3-ium trifluoromethanesulfonate (FM073)

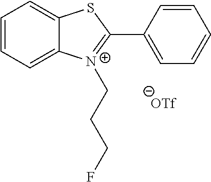 [SMILES: FCCC[n+]1c(-c2ccccc2)sc2ccccc21]

The title compound was synthesised using method (c) above and isolated in 20% yield (crude).

¹H NMR (400 MHz, DMSO-d₆) δ 8.51 (1H, dd, J=8.2, 1.2 Hz), 8.43 (1H, d, J=8.6 Hz), 7.95 (1H, ddd, J=8.6, 7.3, 1.3 Hz), 7.90-7.69 (6H, m), 4.78-4.70 (2H, m), 4.49-4.33 (2H, m), 2.30-2.14 (2H, m).

¹⁹F NMR (376 MHz, DMSO-d₆) δ −77.79.

3-(3-Iodopropyl)-2-phenyl-2,3-dihydrobenzo[d]thiazole (FM069)

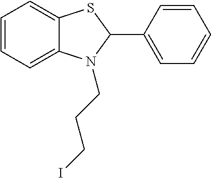 [SMILES: ICCCN1c2ccccc2SC1c1ccccc1]

The title compound was synthesised using method (d) above and isolated in 56% yield.

¹H NMR (400 MHz, DMSO-d₆) δ 7.60-7.52 (2H, m, Ar—CH), 7.51-7.40 (3H, m, Ar-CH), 7.14-7.01 (2H, m, Ar—CH), 6.76-6.68 (1H, m, Ar-CH), 6.64 (1H, d, J=7.9 Hz, Ar-CH), 6.41 (1H, s, CH), 3.27 (2H, q, J=7.0 Hz, Ar—CH₂), 3.02 (2H, ddd, J=14.6, 8.8, 5.8 Hz, CH₂), 2.10-1.90 (2H, m, CH₂).

13C NMR (101 MHz, DMSO-d₆) δ 146.63 (qC), 140.60 (qC), 128.76 (CH), 128.70 (CH), 126.90 (CH), 125.76 (CH), 124.63 (qC), 121.20 (CH), 118.63 (CH), 107.16 (CH), 72.30 (CH), 46.60 (CH₂), 29.38 (CH₂), 4.97 (CH₂).

3-(3-Fluoropropyl)-2-phenyl-2,3-dihydrobenzo[d]thiazole (FM074)

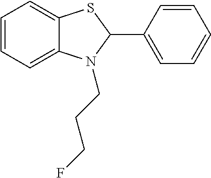 [SMILES: FCCCN1c2ccccc2SC1c1ccccc1]

The title compound was synthesised using method (d) above and isolated in 52% yield.

¹H NMR (400 MHz, DMSO-d₆) δ 7.60-7.51 (m, 2H, Ar—CH), 7.50-7.38 (m, 3H, Ar—CH), 7.12-7.01 (m, 2H, Ar—CH), 6.70 (td, J=7.5, 1.1 Hz, 1H, Ar—CH), 6.58 (dd, J=8.0, 1.1 Hz, 1H, Ar—CH), 6.41 (s, 1H, CH), 4.59-4.38 (m, 2H, CH₂), 3.03 (ddd, J=14.7, 8.8, 6.0 Hz, 2H, CH₂), 1.97-1.71 (m, 2H, CH₂).

¹³C NMR (101 MHz, DMSO-d₆) δ 146.64 (qC), 140.56 (qC), 128.76 (CH), 128.70 (CH), 126.91 (CH), 125.80 (CH), 124.60 (qC), 121.18 (CH), 118.55 (CH), 107.06 (CH), 82.55 and 80.94 (CF), 72.22 (CH₂), 42.19 (CH₂), 26.51 (CH₂).

19F NMR (376 MHz, DMSO-d₆) δ-106.93.

2-(4-Methoxyphenyl)benzo[d]thiazole (FM054)

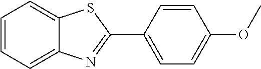 [SMILES: COc1ccc(-c2nc3ccccc3s2)cc1]

The title compound was synthesised using method (a) above and isolated in 63% yield.

¹H NMR (400 MHz, DMSO-d₆) δ: 8.13-8.07 (1H, m, ArH), 8.07-7.98 (3H, m, ArH), 7.52 (1H, ddd, J=8.3, 7.2, 1.3 Hz, ArH), 7.43 (1H, ddd, J=8.3, 7.2, 1.2 Hz, ArH), 7.15-7.08 (2H, m, ArH), 3.85 (3H, s, CH₃).

¹³C NMR (101 MHz, DMSO-d₆) δ 167.01 (qC), 161.73 (qC), 153.61 (qC), 134.17 (qC), 128.83(CH), 126.48 (CH), 125.46 (qC), 125.07 (CH), 122.42 (CH), 122.15 (CH), 114.70 (CH), 55.45 (CH₃).

3-(3-Floropropyl)-2-(4-methoxyphenyl)benzo[d]thiazol-3-ium trifluoromethanesulfonate (FM107)

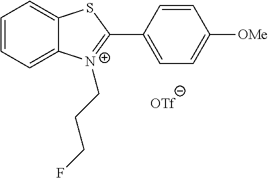 [SMILES: COc1ccc(-c2sc3ccccc3[n+]2CCCF)cc1]

The title compound was synthesised using method (c) above and used for next step without purification.

3-(3-Floropropyl)-2-(4-methoxyphenyl)-2,3-dihydrobenzo[d]thiazole (FM108)

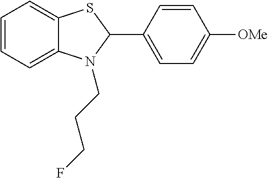 [SMILES: COc1ccc(C2Sc3ccccc3N2CCCF)cc1]

The title compound was synthesised using method (d) above and isolated in 58% yield.

¹H NMR (400 MHz, DMSO-d₆) δ 7.56-7.48 (2H, m), 7.09-6.99 (4H, m), 6.68 (1H, td, J=7.5, 1.1 Hz), 6.63 (1H, dd, J=7.9, 1.0 Hz), 6.34 (1H, s), 4.69-4.39 (m, 2H, CH₂), 3.88 (3H, s), 3.01 (m, 2H, CH₂), 1.99-1.75 (m, 2H, CH₂).

13C NMR (101 MHz, DMSO-d₆) δ 159.61 (qC), 146.58 (qC), 132.06 (qC), 128.59 (CH), 125.65 (CH), 124.73 (CH), 121.16 (CH), 118.52 (CH), 114.04 (CH), 107.07 (CH), 72.29 (CH), 82.56 and 80.95 (CF), 72.21 (CH₂), 55.15 (CH₃), 42.20 (CH₂), 26.53 (CH₂).

3-(3-Iodopropyl)-2-(4-methoxyphenyl)benzo[d]thiazol-3-ium trifluoromethanesulfonate (FM071)

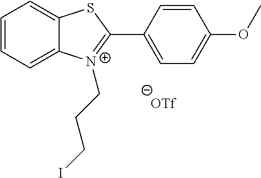 [SMILES: COc1ccc(-c2sc3ccccc3[n+]2CCCI)cc1]

The title compound was synthesised using method (c) above and used for next step without purification.

3-(3-IodopropyI)-2-(4-methoxyphenyl)-2,3-dihydrobenzo[d]thiazole (FM083)

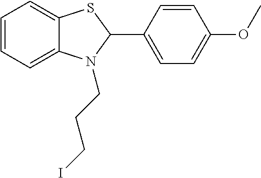 [SMILES: COc1ccc(C2Sc3ccccc3N2CCCI)cc1]

The title compound was synthesised using method (d) above and isolated in 36% yield.

¹H NMR (400 MHz, DMSO-d₆) δ 7.55-7.45 (2H, m), 7.08-6.97 (4H, m), 6.69 (1H, td, J=7.5, 1.1 Hz), 6.60 (1H, dd, J=7.9, 1.0 Hz), 6.37 (1H, s), 3.82 (3H, s), 3.30-3.20 (2H, m), 3.04-2.84 (2H, m), 2.03-1.83 (2H, m).

13C NMR (101 MHz, DMSO-d₆) δ 159.57 (qC), 146.57 (qC), 132.00 (qC), 128.49 (CH), 125.70 (CH), 124.71 (CH), 121.14 (CH), 118.51 (CH), 114.01 (CH), 107.06 (CH), 72.26 (CH), 55.14 (CH₃), 46.25 (CH₂), 29.21 (CH₂), 5.02 (CH₂).

2. Radiochemistry

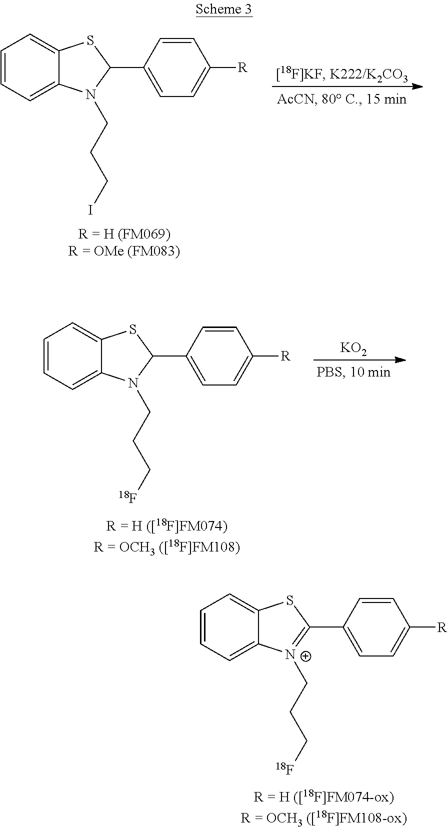

As shown in Scheme 3 above, precursor compounds were radiolabelled to form ¹⁸F-labelled compounds. These ¹⁸F-labelled compounds were then oxidised to obtain their oxidised analogues.

Radiolabelling

Radiofluorination was performed by the nucleophilic substitution of the alkyl iodine in the precursor compounds, FM069 and FM083.

[¹⁸F]Fluoride (˜200-1200 MBq) in water was trapped in a carbonated QMA cartridge (Waters Sep-Pak light) pre-treated with water (10 mL), and released with 1.0 mL of Kryptofix 222 and potassium carbonate mixture (30:15 mM) dissolved in acetonitrile/water (85:15). After removing the solvents by heating at 110° C. under a stream of nitrogen for 15 min, azeotropic distillation with anhydrous acetonitrile (400 μL) was repeated twice at 90° C. for another 15 min. A solution of precursor (16 μmol) in anhydrous acetonitrile (400 μL) was then added and heated at 80° C. for 15 min in a closed Wheaton vial. The reaction was cooled to room temperature and quenched by addition of water (100 μL) and purified by semi-preparative HPLC. The radiolabelled product was collected from the HPLC column and diluted to 10% acetonitrile in water. It was trapped onto a Sep-Pak C-18 light cartridge (pre-activated with 5 mL methanol followed by 5 mL water). The cartridge was washed with 2 mL of water and the product was then released with 1 mL of pure ethanol. The isolated tracer in ethanol was used for subsequent assays.

[¹⁸F]-(3-fluoropropyl)-2-phenyl-2,3-dihydrobenzo[d]thiazole (¹⁸F-FM074) and [¹⁸F]3-(3-fluoropropyl)-2-(4-methoxyphenyl)-2,3-dihydrobenzo[d]thiazole (¹⁸F-FM108) were each purified with a ZORBAX column (3005B-C₁₈, semi-preparative 9.4×250 mm, 5 pm) using AcCN and water as the mobile phase, at a flow rate of 3 mL/min. The following gradient was used: from 50% to 90% AcCN in 15 min; kept at 90% AcCN for 10 min; from 90 to 50 AcCN in 5 min.

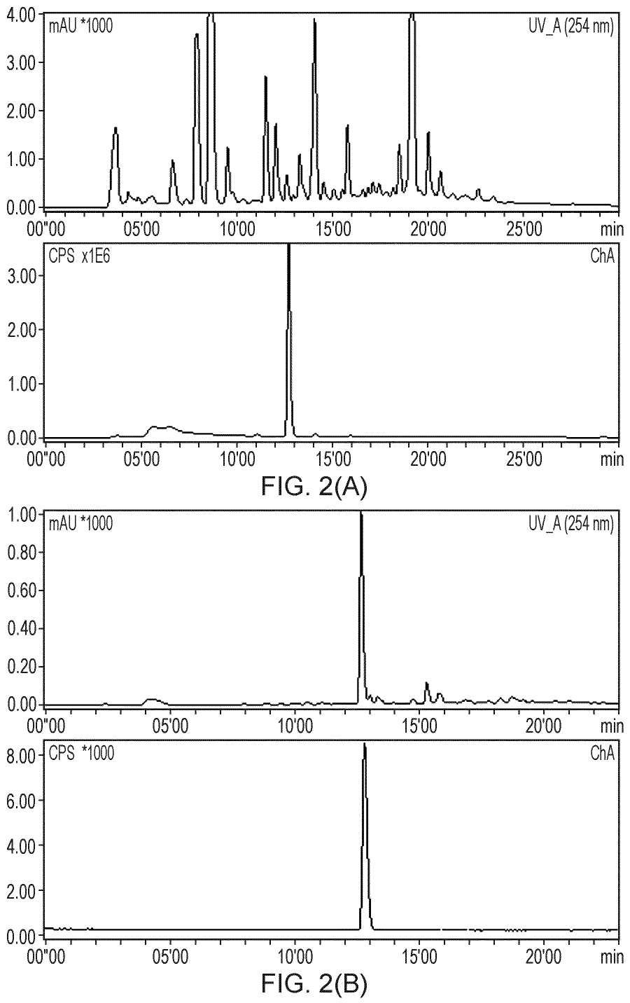

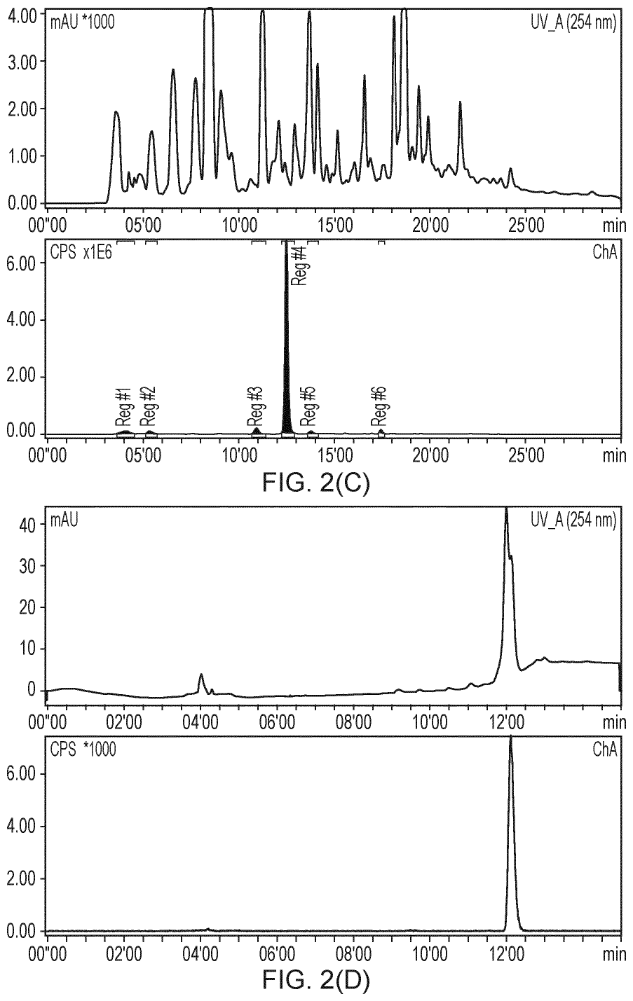

The radiosynthesis of ¹⁸F-FM074 and ¹⁸F-FM108 was achieved with near quantitative radiochemical conversion as indicated by the radioHPLC (FIGS. 2(A) and 2(C)) and with non-decay corrected isolated radiochemical yields of 10±3% (n=10) and 18±(n=5), respectively, from the end of bombardment until formulation for injection.

The identity of ¹⁸F-FM074 and ¹⁸F-FM108 was confirmed by HPLC co-elution with the corresponding nonradioactive reference compounds (FIGS. 2(B) and 2(D)).

Molar Activity

The molar activity of ¹⁸F-FM074 and ¹⁸F-FM108 was determined. Molar activity (Am) is the measured radioactivity per mole of compound, commonly measured in Bq/mol or GBq/μmol.

The molar activity of ¹⁸F-FM074 and ¹⁸F-FM108 was measured as 168±39 GBq/μmol (n =3) and 136±17 GBq/μmol (n=3), respectively, when started with around 1 GBq of ¹⁸F-fluoride.

Due to the ¹⁸F radiolabelling being achieved with near quantitative radiochemical yield, the ¹⁸F tracers can be produced with high molar activity.

Oxidation

The oxidised analogues, ¹⁸F-FM074-Ox and ¹⁸F-FM108-Ox, were obtained by reacting ¹⁸F-FM074 or ¹⁸F-FM108 (˜50 MBq, 1000 μL in PBS containing 10% ethanol) with potassium superoxide (˜10 mg) until full oxidation was observed by radio-HPLC.

3. LogD Measurement

The lipophilicity of ¹⁸F-FM074 and ¹⁸F-FM074-Ox was determined by measuring their logD values using a variation of the conventional shake-flask method, namely a conventional partition method between 1-octanol and phosphate buffered saline (PBS), pH 7.4. The 1-octanol was saturated with PBS before use. The radiotracer (1 μL) in ethanol was added to a mixture of PBS (200 μL) and 1-octanol (200 μL) in a 1.5 mL Eppendorf vial (n =6). The vial was sealed and shaken, then centrifuged at 3000 g for 10 min. A 100 μL aliquot from each layer was drawn was added to separate tubes. The radioactivity content of each fraction was measured in a gamma counter. The LogD_{oct/PBS} as calculated as follows: log [(cpm in the 1-octanol layer-cpm 1-octanol blank)/(cpm in the PBS layer-cpm pbs blank)].

¹⁸F-FM074 has a logD of 1.00±0.08 (n=12) and its oxidised form ¹⁸F-FM074-Ox has a significantly lower logD of −1.00±0.04 (n=6). These suggest that ¹⁸F-FM074 would enter the cells by passive diffusion, while ¹⁸F-FM074-Ox is much less cell membrane permeable. The log D of ¹⁸F-FM108 was also determined as 0.83±0.12 (n=12).

4. Stability Tests

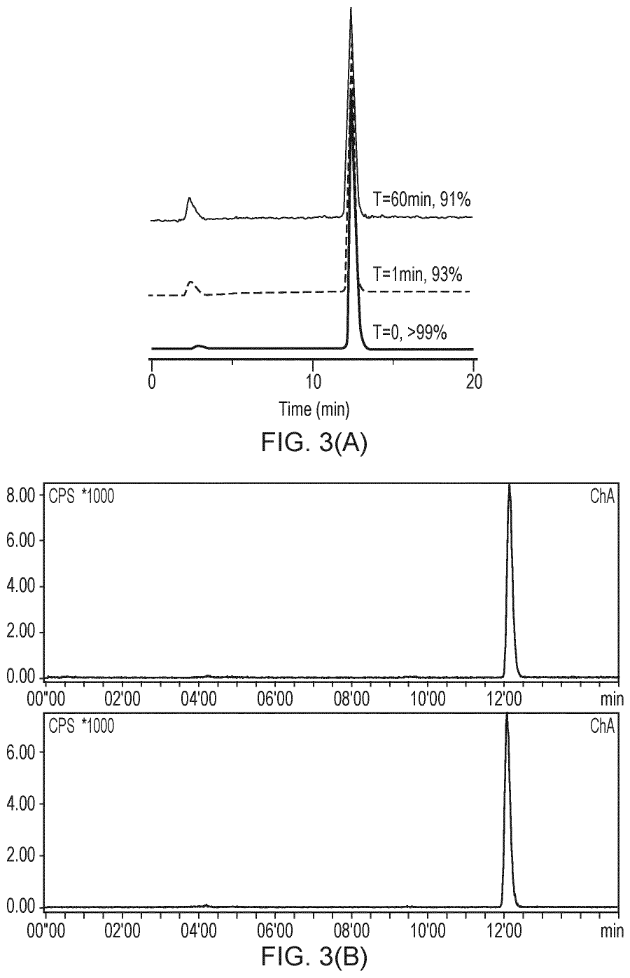

Stability tests monitored by HPLC indicate that ¹⁸F-FM074 is stable in both pure ethanol and 1% ethanol in PBS in the presence of ascorbic acid (0.01 mg/mL) for 4 h (not tested for longer). When incubated in rat serum at 37° C., 90% of ¹⁸F-FM074 was intact in 1 hour (FIG. 3(A)). The stability of ¹⁸F-FM108 in PBS was also determined and it was stable in 30 minutes (FIG. 3(B)).

5. Chemoselectivity Studies

We investigated the chemoselectivity of ¹⁸F-FM074 and ¹⁸F-FM108 towards various ROS in vitro.

The different oxidants were prepared in PBS (900 μL). The ¹⁸F-labelled compound was formulated as 0.5-2 MBq in 100 μL ethanol and added to the oxidant PBS solution. The final reaction mixture contains the ¹⁸F-labelled compound (0.5-2 MBq) in the presence of 100 μM oxidant in PBS with 10% ethanol (final volume 1000 μL). The reaction was kept at room temperature for 5 min, after which it was injected into the HPLC for analysis.
Superoxide (O₂⁻): The ¹⁸F-labelled compound in ethanol (100 μL) was added to PBS (900 μL) and the resulting solution was then added to a vial containing solid KO₂ (1 mg), to make up a reaction mixture with 10 mM O₂⁻.
Hydrogen peroxide (H₂₀₂): H₂O₂ (10 mM stock solution, 10 μL) was diluted in PBS (890 μL) followed by addition of the ¹⁸F-labelled compound in ethanol (100 μL), to a final concentration of 100 μM.
Hydroxyl radical (⋅OH): hydroxyl radicals were generated in situ by reacting hydrogen peroxide (H_{202, 100} μM) with iron II (1000 pM). FeSO₂.7H₂O (5 mM stock, 200 μL) was diluted in PBS (690 μL) and H₂O₂ (10 mM stock, 10 μL) was added, followed by the ¹⁸F-labelled compound in 100 μL ethanol.
Iron (II) sulfate heptahydrate (Fe²⁺) (control): FeSO₂.7H₂O (5 mM stock, 200 μL) was diluted to a final concentration of 1000 μM in PBS (700 μL), and the ¹⁸F-labelled compound in 100 μL ethanol was added after.
tert-butyl hydroperoxide (TBHP): TBHP (10 mM stock, 10 μL) was diluted in PBS (890 μL) followed by addition of the ¹⁸F-labelled compound in ethanol, to a final concentration of 100 μM.
Tert-butoxy radical (t-BuO⋅): t-butoxy radicals were generated in situ by reacting tert-butyl hydroperoxide (TBHP, 100 μM) with Fe²⁺ (1000 μM). FeSO_{2.}7H₂O (5 mM stock, 200 μL) was diluted in PBS (690 μL) and TBHP (10 mM stock, 1 μL) was added, followed by the ¹⁸F-labelled compound in ethanol.
Peroxynitrite (ONOO⁻): peroxynitrite was generated in situ by spontaneous decomposition of 3-morpholinosydnomine (SIN-1) in solution. SIN-1 (5 mM stock, 1 μL) was diluted in PBS (989 μL) containing the ¹⁸F-labelled compound in ethanol (10 μL), to give a final concentration of 50 μM ONOO⁻ in solution.
Nitric oxide (NO): NO was generated in situ by the NO-donor drug diethylamine NONOate (DEA/NO). A 33 mM stock solution of DEA/NO in 10 mM NaOH was used. 1 μL was diluted in PBS (990 μL) containing the ¹⁸F-labelled compound in ethanol (10 μL), to give a final concentration of 33 μM DEA/NO in solution. The reaction was allowed to take place for 32 min at room temperature (˜20° C.) to allow DEA/NO to decompose into diethylamine and nitric oxide radical (NO) at a concentration of 50 μM, before HPLC analysis.

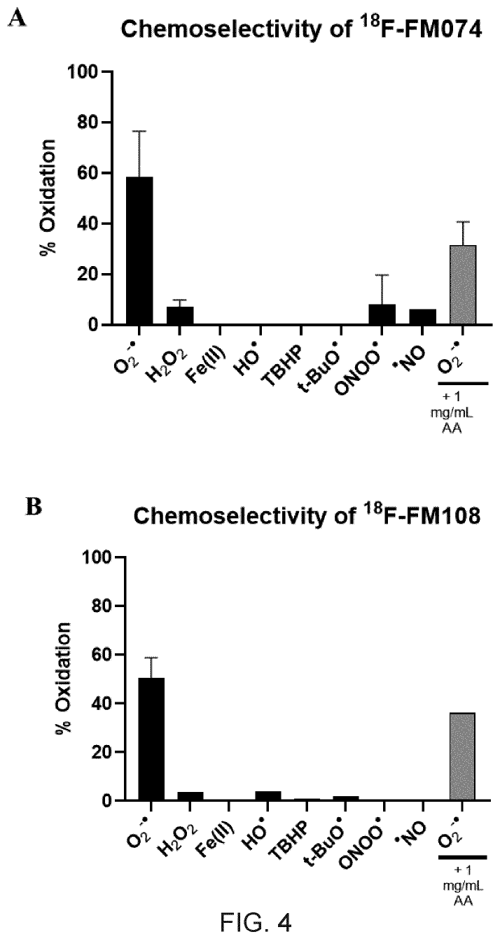

¹⁸F-FM074 showed rapid (in 5 min) and selective oxidation 58±8% (n=4) by superoxide (spontaneous decomposition of 1 mg/mL KO₂ in solution) compared to other endogenous ROS such as hydroxyl radical (⋅OH), hydrogen peroxide (H₂O₂), nitric oxide (⋅NO), and peroxynitrite (ONOO⁻) (either no oxidation or less than 10%) in vitro. In addition, the ¹⁸F-FM074 oxidation by superoxide (K02) can be partially inhibited to 31±5% (n=2) in the presence of ascorbic acid (1 mg/mL) (FIG. 4(A)). In contrast, ¹⁸F-FM074 had little or no reactivity to other biologically relevant ROS.

¹⁸F-FM108 showed rapid (in 5 min) and selective oxidation 54±4% (n=3) by superoxide (spontaneous decomposition of 1 mg/mL KO₂ in solution). In addition, the ¹⁸F-FM108 oxidation by superoxide (KO₂) can be partially inhibited to 38% (n=1) in the presence of (ascorbic acid, 1 mg/mL) (FIG. 4(B)). In contrast, ¹⁸F-FM108 had little or no reactivity to other biologically relevant ROS.

When [¹⁸F]FM074 was incubated with xanthine oxidase, xanthine (1 mM) and catalase in PBS buffer at 37° C. for 5 min, 53% of oxidised [¹⁸F]FM074 was observed in the radioHPLC chromatogram. This indicates that [¹⁸F]FM074 can be oxidised by superoxide generated by the xanthine oxidase/xanthine system, providing an in vitro biological evaluation of [¹⁸F]FM074 towards ROS.

6. In Vivo Experiments

All experiments were performed in accordance with the Animals (Scientific Procedures) Act 1986 under Project Licence numbers PPL P96678ED7 and PPL 70/8482. Male wistar rats were obtained from Envigo Ltd. and went through a 7-day acclimatisation period at the Biological Services Unit at St. Thomas' Hospital prior to any experiments.

6.1 Study in Healthy C₅₇B16 Mice

Wild type C₅₇BL/6J mice (male, 26±2 g) were injected intravenously via the tail vein with [¹⁸F]FM074 (1.2±1.1 MBq) in PBS containing sodium ascorbate (0.01 mg/mL). The animals were sacrificed either 3 min post-injection or after a 60 min PET (n=3/group). Organs and tissues of interest were harvested, weighed and the radioactivity measured in a gamma counter. Organ uptake was calculated as percentage injected dose per gram of tissue mass (% ID/g). Data are reported as mean±SD.

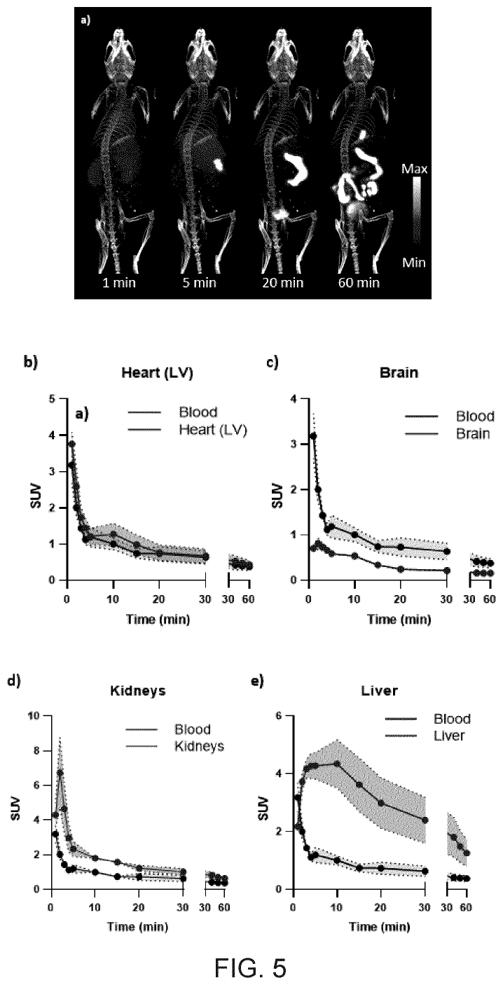

The above PET imaging study in healthy C₅₇B16 mice (n=3) showed that ¹⁸F-FM074 has fast blood clearance and excellent tissue penetration. Regions of interest (ROIs) were drawn on the major organs and the PET signal quantified in the form of time-activity curves using standard uptake values (SUV). ¹⁸F-FM074 is rapidly taken up by the heart and brain, and no non-specific background retention is observed. The radiotracer is excreted by both renal and hepatobilliary routes in less than 1 hour (FIG. 5).

A common concern with ¹⁸F-radiotracers is defluorination in vivo, leading to deposition of ¹⁸F-fluoride in the bones. In our study, bone uptake was minimal after 1 hour, indicating the radiotracer is stable to defluorination in vivo.

6.2 Biodistribution in Healthy Rats

Biodistribution studies in healthy rats were performed.

Male Wistar rats (290-355 g) were placed in a warm box at 37° C. before being transferred to an anaesthesia induction box under an O₂ flow rate of 1 L/min with isofluorane levels of 5%. The anaesthetised rat was transferred to an anaesthesia mask with an O₂ flow rate of 1 L/min with isofluorane levels of 2.5%. The radiotracer (0.5-2.2 MBq, 200-600 μL in maximum 5% EtOH in PBS) was injected intravenously via a cannula inserted into a tail vein. The animals were sacrificed by cervical dislocation at 1, 5, and 30 min post-injection (n=3/group). The heart was immediately removed and rinsed with saline to washout the blood. Organs of interest as well as blood and urine were collected and the radioactivity measured in a gamma counter.

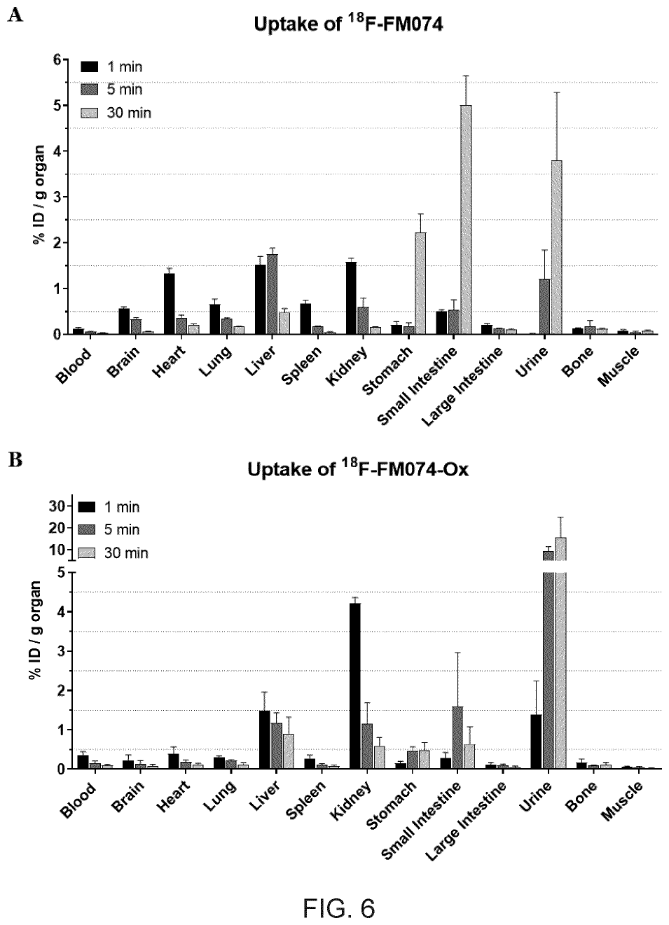

The biodistribution studies in healthy Wistar rats showed rapid initial uptake of ¹⁸F-FM074 by major organs including the brain, heart, lung, and spleen, and fast clearance from the blood pool at 1 min post-injection (FIG. 6(A)). The radiotracer was cleared from these organs in under 30 min through hepatic and renal metabolism, and excreted to the small intestine and urine. In addition, ¹⁸F-FM074 had little uptake in both bone and muscle within 30 min.

Biodistribution studies were also performed with oxidized radiotracer ¹⁸F-FM074-Ox. ¹⁸F-FM074-Ox shows significantly reduced uptake in major organs and was quickly cleared by the kidneys and excreted through the urine (FIG. 6(B)).

Rat Model of Doxorubicin-Induced Cardiotoxicity

To assess whether ¹⁸F-FM074 would have increased uptake in tissues under oxidative stress, PET imaging was performed in a rat model of Doxorubicin-induced cardiotoxicity. Several studies have indicated that ROS generation is a key regulatory mechanism in Doxorubicin-induced cardiotoxicity.

Wistar rats were randomly divided into two groups and were subjected to the implantation of an Alzet Osmotic minipump containing either Doxorubicin (30 mg/kg) in saline (n=6) or saline only (n=4) as control. The minipump releases the drug at a constant rate over seven days, after which PET/CT imaging was performed.

Echocardiography

In order to assess cardiac function all rats were subjected to a cardiac ultrasound (Vevo 770™, VisualSonics) one day prior to osmotic pump implantation and a second ultrasound one day prior to PET/CT imaging. Rats were anaesthetized with 2% isoflurane in 100% oxygen and maintained at 37° C. via a homoeothermic platform and rectal thermometer. High-resolution parasternal left ventricle (LV) long axis M-mode and B-mode images were obtained using a RMV710B transducer. Images were analysed offline using Vevo Software to determine LV function.

Minipump Implantation

Male Wistar rats (280-300 g) were used in all experiments. Subcutaneous 7-day osmotic pumps (Alzet), containing either Doxorubicin (Cambridge Bioscience, 30 mg/kg cumulative dose) or vehicle (sterile 0.9% NaCl), were inserted into rats under 2% isoflurane in 100% oxygen.

Doxorubicin (30 mg/kg) or only vehicle (saline) was delivered to the animals using the osmotic minipump for seven days, after which PET/CT imaging was performed.

PET/CT Imaging

Animals were placed in a warm box at 37° C. before being transferred to an anaesthesia induction box under an 02 flow rate of 1 L/min with isofluorane levels of 5%. The anaesthetised rats were transferred to the PET bed and kept under anaesthesia with an O₂ flow rate of 1 L/min with isofluorane levels of 2.5%. The radiotracer (0.5-2.2 MBq, 200-600 μL in maximum 5% EtOH in PBS) was injected intravenously via a cannula inserted into a tail vein. The injection start time coincided with the start of the PET acquisition in order to obtain dynamic tracer uptake information. The PET scans were acquired for 30 min, followed by a CT scan, after which the animals were sacrificed and organs harvested as for the biodistribution protocol. The PET data was analysed using VivoQuant.

Results

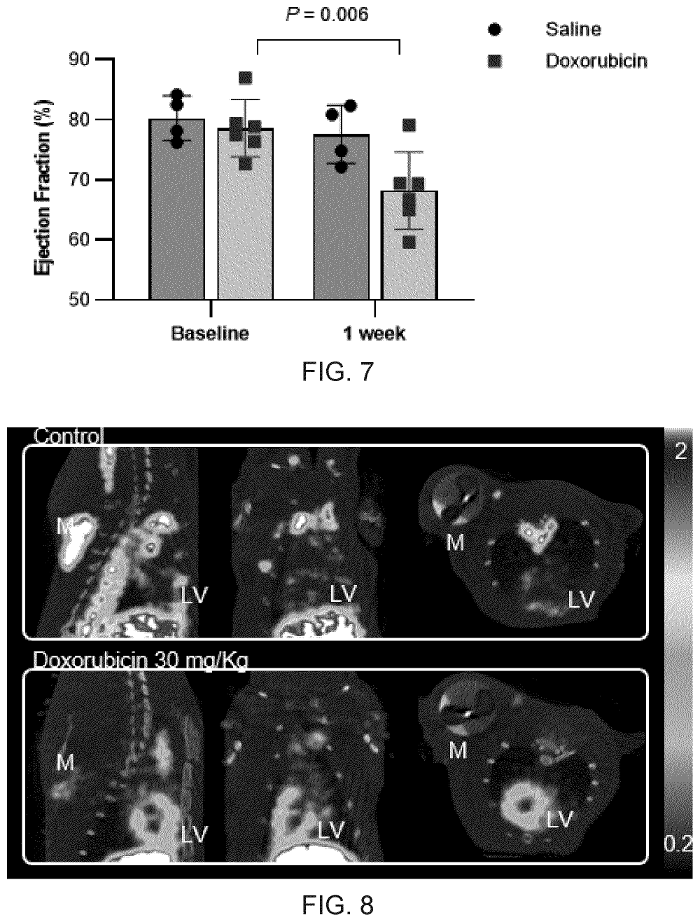

Cardiac function was monitored by ultrasound, and rats treated with Doxorubicin showed statistically significant reduced (15%) left ventricle ejection fraction on day 6 compared to baseline on day 0, but no significant difference was found between Doxorubicin treated and control groups (FIG. 7). These data indicate that ultrasound cannot detect the Doxorubicin-induced damage to the heart of treated animals.

Subsequently, both groups were subjected to 30 min PET/CT scans with chest cavity in the field of view on day 7 of the treatment. (FIG. 8) The PET images were analysed into three segments: from 0 to 3 min, 3 to 10 min, and 10 to 30 min post-injection of ¹⁸F-FM074.

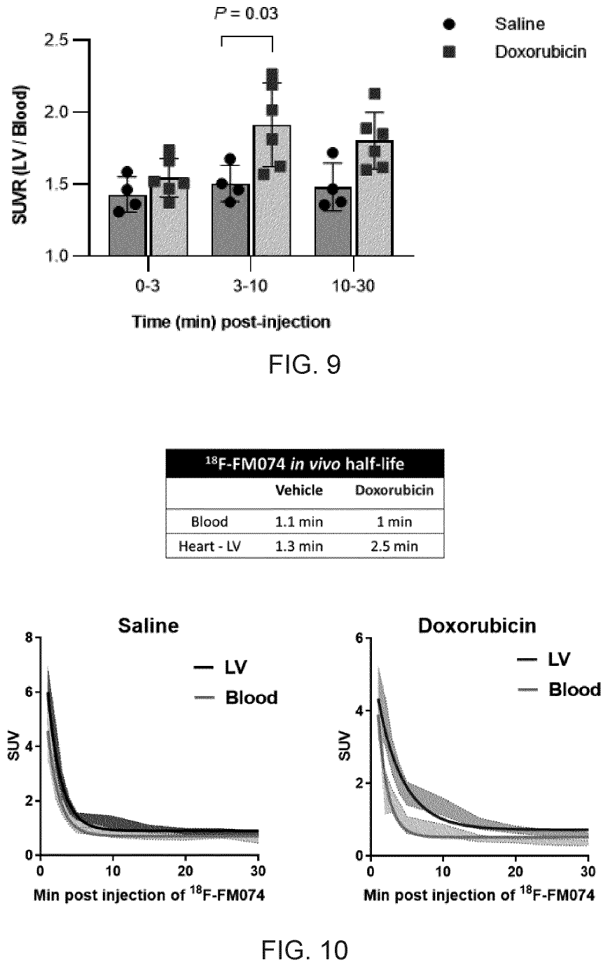

The uptake of ¹⁸F-FM074 in the left ventricle (LV) and blood pool inside the myocardium were quantified and the ratio of the standard uptake values for LV to blood (SUV) was used to account for tracer bioavailability. Between 3-10 min post-injection radioactivity uptake was significantly (p<0.05) higher for the Doxorubicin-treated rats (FIG. 9). However, due to the fast clearance of ¹⁸F-FM074, no significant difference in the heart uptake was found ex vivo after the PET/CT scan. In some PET scans tracer uptake was visible in the area where the minipump incision was made, which we hypothesize could be due to inflammation and poor perfusion. Additionally, high uptake was also observed in the liver, although no significant difference was found between treated and control groups.

The dynamic profile of the radiotracer uptake and washout in the left ventricle (LV) and blood pool inside the myocardium were also assessed resulting in time-activity curves (standard uptake values vs time) of ¹⁸F-FM074 uptake in the left ventricle (LV) and blood pool inside the myocardium in the rat model of Doxorubicin-induced cardiotoxicity (FIG. 10). The washout rate from the LV is two-fold slower in the LV of Doxorubicin treated animals compared to that of the control.

The PET imaging data indicates that using [¹⁸F]FM074 can detect the Doxorubicin-induced damage to the heart of treated animals. The observed increased uptake in the hearts of Doxorubicin-treated rats when compared to controls highlights the potential of ¹⁸F-FM074 as a PET tracer to noninvasively detect oxidative stress in vivo.

### REFERENCES

Sabharwal S S, Schumacker P T. Mitochondrial ROS in cancer: initiators, amplifiers or an Achilles' heel? Nature Reviews Cancer. 2014; 14(11):709-21.

Finkel T, Holbrook N.J. Oxidants, oxidative stress and the biology of ageing. nature. 2000; 408(6809):239.

Maioli N, Zarpelon A, Mizokami S, Calixto-Campos C, Guazelli C, Hohmann M, et al. The superoxide anion donor, potassium superoxide, induces pain and inflammation in mice through production of reactive oxygen species and cyclooxygenase-2. Brazilian Journal of Medical and Biological Research. 2015; 48(4):321-31.

Touyz R. Reactive oxygen species and angiotensin II signaling in vascular cells: implications in cardiovascular disease. Brazilian Journal of Medical and Biological Research. 2004; 37(8):1263-73.

Wang T, Qin L, Liu B, Liu Y, Wilson B, Eling T E, et al. Role of reactive oxygen species in LPS-induced production of prostaglandin E2 in microglia. Journal of neurochemistry. 2004; 88(4):939-47.

Winterbourn C C. Reconciling the chemistry and biology of reactive oxygen species. Nature chemical biology. 2008; 4(5):278.

Deavall D G, Martin E A, Horner J M, Roberts R. Drug-induced oxidative stress and toxicity. Journal of toxicology. 2012; 2012.

Singal P K, Iliskovic N. Doxorubicin-induced cardiomyopathy. New England Journal of Medicine. 1998; 339(13):900-5.

Swain S M, Whaley F S, Ewer M S. Congestive heart failure in patients treated with doxorubicin: a retrospective analysis of three trials. Cancer: Interdisciplinary International Journal of the American Cancer Society. 2003; 97(11):2869-79.

Zhang S, Liu X, Bawa-Khalfe T, Lu L-S, Lyu Y L, Liu L F, et al. Identification of the molecular basis of doxorubicin-induced cardiotoxicity. Nature medicine. 2012; 18(11):1639.

Kim S-Y, Kim S-J, Kim B-J, Rah S-Y, Chung SM, Im M-J, et al. Doxorubicin-induced reactive oxygen species generation and intracellular Ca2+ increase are reciprocally modulated in rat cardiomyocytes. Experimental & molecular medicine. 2006; 38(5):535.

Kurz E U, Douglas P, Lees-Miller S P. Doxorubicin activates ATM-dependent phosphorylation of multiple downstream targets in part through the generation of reactive oxygen species. Journal of Biological Chemistry. 2004; 279(51):53272-81.

Chu W, Chepetan A, Zhou D, Shoghi K I, Xu J, Dugan L L, et al. Development of a PET radiotracer for non-invasive imaging of the reactive oxygen species, superoxide, in vivo. Organic & biomolecular chemistry. 2014; 12(25):4421-31.

Hou C, Hsieh C-J, Li S, Lee H, Graham T J, Xu K, et al. Development of a positron emission tomography radiotracer for imaging elevated levels of superoxide in neuroinflammation. ACS chemical neuroscience. 2017; 9(3): 578-86.

Lynn M A, Carlson L J, Hwangbo H, Tanski J M, Tyler L A. Structural influences on the oxidation of a series of 2-benzothiazoline analogs. Journal of Molecular Structure. 2012; 1011:81-93.

Tang B, Zhang L, Zhang L-I. Study and application of flow injection spectrofluorimetry with a fluorescent probe of 2-(2-pyridil)-benzothiazoline for superoxide anion radicals. Analytical biochemistry. 2004; 326(2):176-82.

Kil H J, Lee I-SH. Primary Kinetic Isotope Effects on Hydride Transfer from Heterocyclic Compounds to NAD+ Analogues. The Journal of Physical Chemistry A. 2009; 113(40):10704-9.

Carroll, V., Michel, B. W., Blecha, J., VanBrocklin, H., Keshari, K., Wilson, D., and Chang, C. J. (2014) A boronate-caged [(1)(8)F]FLT probe for hydrogen peroxide detection using positron emission tomography. J. Am. Chem. Soc. 136, 14742-14745.

Carroll, V. N., Truillet, C., Shen, B., Flavell, R. R., Shao, X., Evans, M. J., VanBrocklin, H. F., Scott, P. J., Chin, F. T., and Wilson, D. M. (2016) [(11)C]Ascorbic and [(11)C]dehydroascorbic acid, an endogenous redox pair for sensing reactive oxygen species using positron emission tomography. Chem. Commun. (Cambridge, U. K.) 52, 4888-4890.

Al-Karmi, S., Albu, S. A., Vito, A., Janzen, N., Czorny, S., Banevicius, L., Nanao, M., Zubieta, J., Capretta, A., and Valliant, J. F. (2017) Preparation of an 18 F-Labeled Hydrocyanine Dye as a Multimodal Probe for Reactive Oxygen Species. Chem. Eur. J. 23, 254-258;

Yang, H., Jenni, S., Colovic, M., Merkens, H., Poleschuk, C., Rodrigo, I., Miao, Q., Johnson, B. F., Rishel, M. J., Sossi, V., Webster, J. M., Benard, F., and Schaffer, P. (2017) ¹⁸F-5-Fluoroaminosuberic Acid as a Potential Tracer to Gauge Oxidative Stress in Breast Cancer Models. J. Nucl. Med. 58, 367-373,

Okamura, T., Okada, M., Kikuchi, T., Wakizaka, H., and Zhang, M. R. (2015) A (11)C-labeled 1,4-dihydroquinoline derivative as a potential PET tracer for imaging of redox status in mouse brain,. J. Cereb. Blood Flow Metab. 35, 1930-1936,

Wilson, A. A., Sadovski, O., Nobrega, J. N., Raymond, R. J., Bambico, F. R., Nashed, M. G., Garcia, A., Bloomfield, P. M., Houle, S., Mizrahi, R., and Tong, J. (2017) Evaluation of a novel radiotracer for positron emission tomography imaging of reactive oxygen species in the central nervous system. Nucl. Med. Biol. 53, 14-20;

Abe, K., Takai, N., Fukumoto, K., Imamoto, N., Tonomura, M., Ito, M., Kanegawa, N., Sakai, K., Morimoto, K., Todoroki, K., and Inoue, O. (2014) In vivo imaging of reactive oxygen species in mouse brain by using [3H]hydromethidine as a potential radical trapping radiotracer. J. Cereb. Blood Flow Metab. 34, 1907-1913;

Takai, N., Abe, K., Tonomura, M., Imamoto, N., Fukumoto, K., Ito, M., Momosaki, S., Fujisawa, K., Morimoto, K., Takasu, N., and Inoue, O. (2015) Imaging of reactive oxygen species using [(3)H]hydromethidine in mice with cisplatin-induced nephrotoxicity. EJNMMI Res. 5, 116;

Abe, K., Tonomura, M., Ito, M., Takai, N., Imamoto, N., Rokugawa, T., Momosaki, S., Fukumoto, K., Morimoto, K., and Inoue, O. (2015) Imaging of reactive oxygen species in focal ischemic mouse brain using a radical trapping tracer [(3)H]hydromethidine. EJNMMI Res. 5, 115;

1. A radiolabelled compound of formula (I): 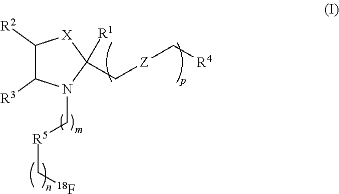
wherein:
X is selected from —O—, —S— or —NR²⁰—;
Z is a double bond or a triple bond;
R¹ is —H or —D;
R² and R³ are linked to form part of an optionally substituted 5- or 6-membered aromatic ring;
R⁴ is selected from —H, optionally substituted alkyl, optionally substituted alkenyl, optionally substituted alkynyl, and or optionally substituted aryl;
R⁵ is selected from optionally substituted alkylene, optionally substituted alkenylene, optionally substituted alkynylene and or optionally substituted arylene;
R²⁰ is selected from —H, alkyl, alkenyl, alkynyl, acyl and or aryl;
n is an integer from 0 to 18;
m is and integer from 0 to 18; and
p is an integer from 0 to 18;
or a pharmaceutically acceptable salt thereof.

2-34. (canceled)

35. The radiolabelled compound of claim 1, wherein X is —S—.

36. The radiolabelled compound of claim 1, wherein Z is a double bond.

37. The radiolabelled compound of claim 1, wherein R¹ is —D.

38. The radiolabelled compound of claim 1, wherein R² and R³ are linked to form part of an optionally substituted 5- or 6-membered aromatic ring selected from: 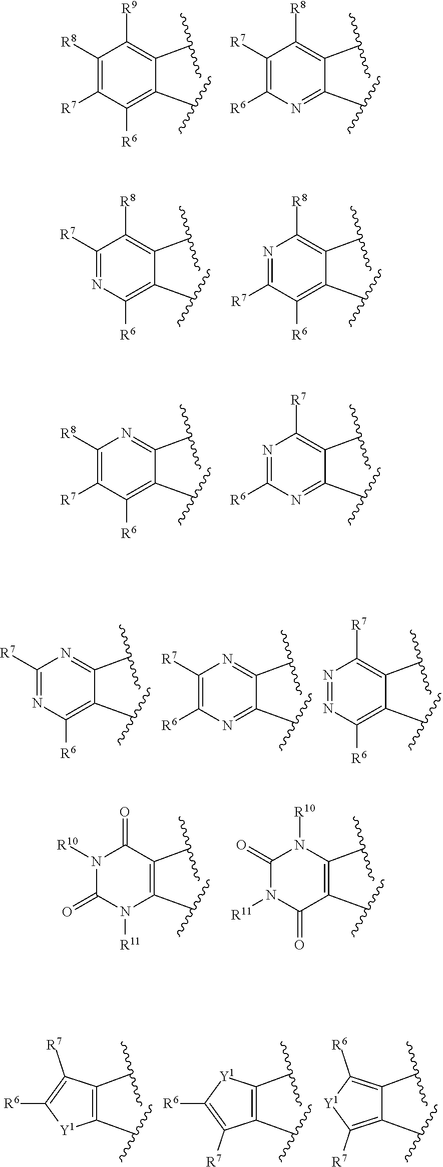
wherein
Y¹ is selected from —O—, —S— or —NR²⁰—;
R⁶, R⁷, R⁸ and R⁹ are independently selected from —H, alkyl, alkenyl, alkynyl, aryl, —CF₃, halogen, —B(OR²⁰)₂, —OR²⁰, —NR²⁰₂, —SR²⁰, —SiR²⁰₃, —SO₃, —SO₃R²⁰, SO₂NR²⁰₂, —S(O)R²⁰, —C(O)R²⁰, —C(O)NR²⁰₂, —CO₂R²⁰, —NO₂, or —CN;
R¹ and R¹¹ are independently selected from —H, alkyl, alkenyl alkynyl, acyl, or aryl; and
R²⁰ is selected from —H, alkyl, alkenyl, alkynyl, acyl or aryl.

39. The radiolabelled compound of claim 1, wherein R² and R³ are linked to form part of a benzene ring.

40. The radiolabelled compound of claim 1, wherein R⁴ is a 5- or 6-membered aromatic moiety selected from: 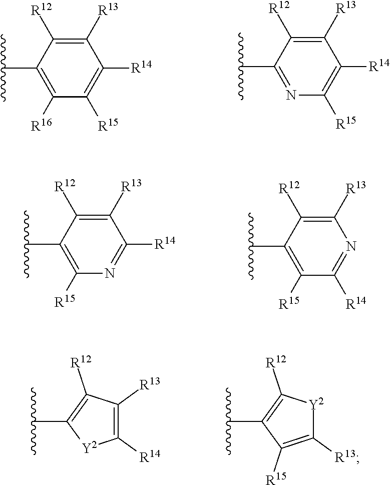
wherein
Y² is selected from —O—, —S— or —NR²⁰—;
R¹², R¹³, R¹⁴, R¹⁵ and R¹⁶ are independently selected from —H, alkyl, alkenyl, alkynyl, aryl,
—CF₃, halogen, —B(OR²⁰)₂, —OR²⁰, —NR²⁰₂, —SR²⁰, —SiR²⁰₃, —SO₃, —SO₃R²⁰, —SO₂NR²⁰₂,
—S(O)R²⁰, —C(O)R²⁰, —C(O)NR²⁰₂, —CO₂R²⁰, —NO₂, or —CN; and
R²⁰ is selected from —H, alkyl, alkenyl, alkynyl, acyl or aryl.

41. The radiolabelled compound of claim 1, wherein R⁴ is phenyl.

42. The radiolabelled compound of claim 1, wherein R⁵ is —(CH₂)_{q}—, wherein q is an integer from 1 to 20.

43. The radiolabelled compound of claim 42, wherein q is 3.

44. The radiolabelled compound of claim 1, wherein R²⁰ is —H or C₁₋₆ alkyl.

45. The radiolabelled compound of claim 1, wherein n is 0; m is 0; or p is 0.

46. A reference compound of formula (II): 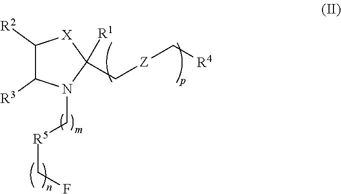
wherein:
X is selected from —O—, —S— or —NR²⁰—;
Z is a double bond or a triple bond;
R¹ is —H or —D;
R² and R³ are linked to form part of an optionally substituted 5- or 6-membered aromatic ring;
R⁴ is selected from —H, optionally substituted alkyl, optionally substituted alkenyl, optionally substituted alkynyl, or optionally substituted aryl;
R⁵ is selected from optionally substituted alkylene, optionally substituted alkenylene, optionally substituted alkynylene or optionally substituted arylene;
R²⁰ is selected from —H, alkyl, alkenyl, alkynyl, acyl or aryl;
n is an integer from 0 to 18;
m is and integer from 0 to 18; and
p is an integer from 0 to 18;
or a pharmaceutically acceptable salt thereof.

47. A precursor compound of formula (III): 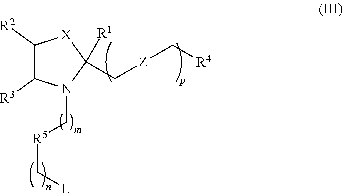
wherein:
X is selected from —O—, —S— or —NR²⁰—;
Z is a double bond or a triple bond;
R¹ is —H or —D;
R² and R³ are linked to form part of an optionally substituted 5- or 6-membered aromatic ring;
R⁴ is selected from —H, optionally substituted alkyl, optionally substituted alkenyl, optionally substituted alkynyl, or optionally substituted aryl;
R⁵ is selected from optionally substituted alkylene, optionally substituted alkenylene, optionally substituted alkynylene or optionally substituted arylene;
R²⁰ is selected from —H, alkyl, alkenyl, alkynyl, acyl or aryl;
n is an integer from 0 to 18;
m is and integer from 0 to 18;
p is an integer from 0 to 18; and
L is a leaving group;
or a pharmaceutically acceptable salt thereof.

48. The precursor compound of claim 47, wherein L is selected from —I, —Br, —Cl, —OTf,
—OMs or —OTs.

49. A pharmaceutical composition comprising the radiolabelled compound of claim 1 and a pharmaceutically acceptable carrier.

50. A process for radiolabelling a precursor compound to form the radiolabelled compound of claim 1, the process comprising:
reacting a precursor compound of formula (III) with nucleophilic fluoride-18 to form the radiolabelled compound of formula (I), in accordance with the following reaction scheme: 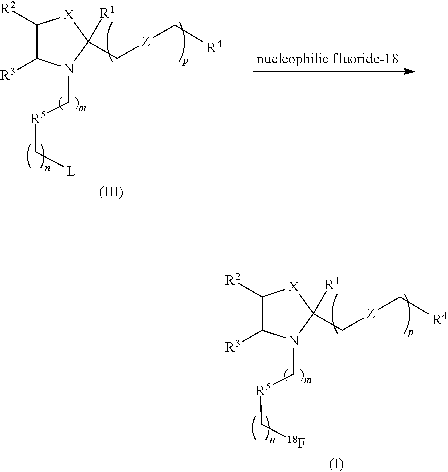
wherein X, Z, R¹, R², R³, R⁴, R⁵, n, m and p are as defined in claim 1; and
L is a leaving group.

51. A process for making the precursor compound of claim 47, the method comprising:
(i) reacting a compound of formula (A) with a compound of formula (E) via a cyclization reaction to form a compound of formula (B);
(ii) reacting the compound of formula (B) with a compound of formula (F) via a nucleophilic addition reaction to form a compound of formula (C); and
(iii) reducing the compound of formula (C) to form a compound of formula (D);
in accordance with the following reaction scheme: 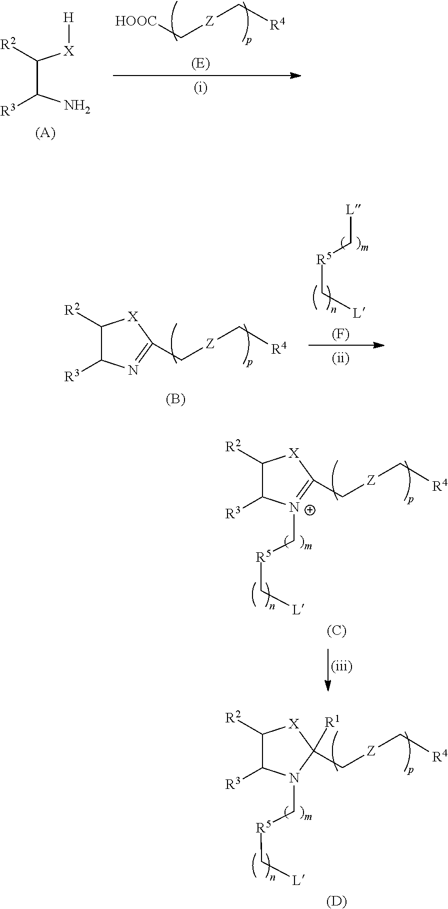
wherein X, Z, R¹, R², R³, R⁴, R⁵, n, m and p are as defined in claim 47;
L′ is a leaving group L as defined in claim 47;
L″ is a leaving group; and
the compound of formula (D) is the precursor compound of formula (III) of claim

47.

52. The process of claim 51, wherein the leaving group L is selected from —I or —Br.

53. The process of claim 51, wherein L″ is a sulfonate ester selected from —OTf, —OMs, or —OTs.
